SEQUENCE LISTING (39 sequences)

SEQ ID NO: 1 - DNA, 25 bp, Monkeypox virus
  source 1..25: mol_type = genomic DNA
ctcgttcgtaatccatttccattag

SEQ ID NO: 2 - DNA, 23 bp, Monkeypox virus
  source 1..23: mol_type = genomic DNA
aacggaacaggaaatacaagaag

SEQ ID NO: 3 - DNA, 30 bp, Monkeypox virus
  source 1..30: mol_type = genomic DNA
actgtatcctcaaacatcctctatcgacgg

SEQ ID NO: 4 - DNA, 22 bp, Monkeypox virus
  source 1..22: mol_type = genomic DNA
gtctccatcatccacatgtatc

SEQ ID NO: 5 - DNA, 24 bp, Monkeypox virus
  source 1..24: mol_type = genomic DNA
ccactggatcaaattcttcgttag

SEQ ID NO: 6 - DNA, 29 bp, Monkeypox virus
  source 1..29: mol_type = genomic DNA
tcgtacacatgttgggagtatgggattcc

SEQ ID NO: 7 - DNA, 23 bp, Monkeypox virus
  source 1..23: mol_type = genomic DNA
gtacgtactatgccaaatgaatc

SEQ ID NO: 8 - DNA, 22 bp, Monkeypox virus
  source 1..22: mol_type = genomic DNA
tacaggctatttacaagatgca

SEQ ID NO: 9 - DNA, 23 bp, Monkeypox virus
  source 1..23: mol_type = genomic DNA
cacgtgttaaacaatgggtgatg

SEQ ID NO: 10 - DNA, 21 bp, Monkeypox virus
  source 1..21: mol_type = genomic DNA
ggtacccacacatacagtatc

SEQ ID NO: 11 - DNA, 21 bp, Monkeypox virus
  source 1..21: mol_type = genomic DNA
cggtacccacacatacagtat

SEQ ID NO: 12 - DNA, 30 bp, Monkeypox virus
  source 1..30: mol_type = genomic DNA
acgtgttaaacaatgggtgatggatacact

SEQ ID NO: 13 - DNA, 26 bp, Monkeypox virus
  source 1..26: mol_type = genomic DNA
attcattcatcattataccattaagt

SEQ ID NO: 14 - DNA, 28 bp, Monkeypox virus
  source 1..28: mol_type = genomic DNA
attcattcatcattataccattaagtgt

SEQ ID NO: 15 - DNA, 30 bp, Monkeypox virus
  source 1..30: mol_type = genomic DNA
agtgtatccatcacccattgtttaacacgt

SEQ ID NO: 16 - DNA, 24 bp, Monkeypox virus
  source 1..24: mol_type = genomic DNA
cagggtgtagaacatgtatttcta

SEQ ID NO: 17 - DNA, 23 bp, Monkeypox virus
  source 1..23: mol_type = genomic DNA
aaacggaagagatttagcaccac

SEQ ID NO: 18 - DNA, 23 bp, Monkeypox virus
  source 1..23: mol_type = genomic DNA
taaacggaagagatttagcacca

SEQ ID NO: 19 - DNA, 23 bp, Monkeypox virus
  source 1..23: mol_type = genomic DNA
taaacggaagagatatagcacca

SEQ ID NO: 20 - DNA, 23 bp, Monkeypox virus
  source 1..23: mol_type = genomic DNA
aaacggaagagatatagcaccac

SEQ ID NO: 21 - DNA, 28 bp, Monkeypox virus
  source 1..28: mol_type = genomic DNA
gacggtgtgagaatatgttccgggacca

SEQ ID NO: 22 - DNA, 21 bp, Monkeypox virus
  source 1..21: mol_type = genomic DNA
agttcccggaggacacgatag

SEQ ID NO: 23 - DNA, 26 bp, Monkeypox virus
  source 1..26: mol_type = genomic DNA
aggacacgatagacaacatagattac

SEQ ID NO: 24 - DNA, 23 bp, Monkeypox virus
  source 1..23: mol_type = genomic DNA
gacggtgtgagaatatgttccgg

SEQ ID NO: 25 - DNA, 23 bp, Monkeypox virus
  source 1..23: mol_type = genomic DNA
tgtgagaatatgttccgggacca

SEQ ID NO: 26 - DNA, 21 bp, Monkeypox virus
  source 1..21: mol_type = genomic DNA
cagatgacgtctccggtagac

SEQ ID NO: 27 - DNA, 22 bp, Monkeypox virus
  source 1..22: mol_type = genomic DNA
cagaagccgtaatctatgttgt

SEQ ID NO: 28 - DNA, 22 bp, Monkeypox virus
  source 1..22: mol_type = genomic DNA
acaacatagattacggcttctg

SEQ ID NO: 29 - DNA, 28 bp, Monkeypox virus
  source 1..28: mol_type = genomic DNA
cgaatacagaagccgtaatctatgttgt

SEQ ID NO: 30 - DNA, 26 bp, Monkeypox virus
  source 1..26: mol_type = genomic DNA
ctttacattttccattggatggtgca

SEQ ID NO: 31 - DNA, 21 bp, Monkeypox virus
  source 1..21: mol_type = genomic DNA
gtctaccggagacgtcatctg

SEQ ID NO: 32 - DNA, 22 bp, Monkeypox virus
  source 1..22: mol_type = genomic DNA
cgtctaccggagacgtcatctg

SEQ ID NO: 33 - DNA, 22 bp, Monkeypox virus
  source 1..22: mol_type = genomic DNA
tctaccggagacgtcatctgtt

SEQ ID NO: 34 - DNA, 22 bp, Monkeypox virus
  source 1..22: mol_type = genomic DNA
cagatgacgtctccggtagacg

SEQ ID NO: 35 - DNA, 22 bp, Monkeypox virus
  source 1..22: mol_type = genomic DNA
aacagatgacgtctccggtaga

SEQ ID NO: 36 - DNA, 22 bp, Monkeypox virus
  source 1..22: mol_type = genomic DNA
ggatacacgtctaccggagacg

SEQ ID NO: 37 - DNA, 22 bp, Monkeypox virus
  source 1..22: mol_type = genomic DNA
cgtctccggtagacgtgtatcc

SEQ ID NO: 38 - DNA, 20 bp, Monkeypox virus
  source 1..20: mol_type = genomic DNA
atacacgtctaccggagacg

SEQ ID NO: 39 - DNA, 20 bp, Monkeypox virus
  source 1..20: mol_type = genomic DNA
cgtctccggtagacgtgtat

(19) United States
(12) Patent Application Publication
(10) Pub. No.: US 2026/0337701 A1
(43) Pub. Date: Oct. 29, 2026

(54) ASSAYS FOR DETECTING MONKEYPOX VIRUS

(71) Applicant: Abbott Laboratories, Abbott Park, IL (US)

(72) Inventors: Gavin Cloherty, Abbott Park, IL (US); Ka-Cheung Luk, Abbott Park, IL (US)

(21) Appl. No.: 19/099,635

(22) PCT Filed: Aug. 3, 2023
(86) PCT No.: PCT/US2023/071560

§ 371 Date: Jan. 29, 2025

Related U.S. Application Data
(60) Provisional application No. 63/395,007, filed on Aug. 4, 2022.
(60) Provisional application No. 63/403,162, filed on Sep. 1, 2022.

(51) Int. Cl. C12Q 1/70; C12Q 1/6888

(52) U.S. Cl. CPC C12Q 1/701; C12Q 1/6888; C12Q 2600/16; C12Q 2600/166

(57) ABSTRACT

The present disclosure relates to materials and methods for amplifying and detecting monkeypox virus in a sample, comprising a variety of combinations of amplification oligonucleotides and oligonucleotide probes. The disclosure also relates to oligonucleotide sequences, kits, and methods for detecting monkeypox virus.

### RELATED APPLICATION INFORMATION

This application claims priority to U.S. Application No. 63/395,007, filed on Aug. 4, 2022 and U.S. Application No. 63/403,162, filed on Sep. 1, 2022, the contents of each of which are herein incorporated by reference.

### SEQUENCE LISTING STATEMENT

The contents of the electronic sequence listing titled “ABBTL_41154_601_ST26.xml” (Size: 35,388 bytes; and Date of Creation: Aug. 1, 2023) is herein incorporated by reference in its entirety.

### FIELD

The present disclosure relates to methods for amplifying target nucleic acid sequences from monkeypox virus and detecting monkeypox infection.

### BACKGROUND

Monkeypox is a viral zoonotic disease caused by monkeypox virus (MPXV), a member of the Orthopoxvirus genus in the family Poxviridae, endemic in West and Central Africa. With the eradication of smallpox and subsequent cessation of smallpox vaccination, monkeypox has emerged as the most important Orthopoxvirus for public health. As of July of 2022, more than 16,000 cases of monkeypox have been reported across more than 70 countries. Severe cases can occur, especially in children, pregnant women, or people with suppressed immune systems, with a fatality ratio of approximately 3-10%. The recent monkeypox outbreaks demonstrated the ability of the virus to exploit new hosts and move globally and quickly.

Monkeypox viruses are divided into two clades or branches, having unique genomic signatures and cause disease of different severity. Viruses sequenced in the current outbreak broadly match those in the typically milder clade. However, methods for detecting the totality of the known genomic signatures of monkeypox viruses are vital for accurately monitoring and detecting present and future monkeypox outbreaks.

### SUMMARY

The disclosure provides a set of reagents, including oligonucleotides, for amplifying and detecting monkeypox virus in a sample. The set can comprise a first amplification oligonucleotide, a second amplification oligonucleotide, and a probe oligonucleotide. In one embodiment, the set may comprise a first amplification oligonucleotide comprising a nucleic acid sequence with at least 70% similarity to SEQ ID NO: 1, a second amplification oligonucleotide comprising a nucleic acid sequence with at least 70% similarity to SEQ ID NO: 2, and a probe oligonucleotide comprising a nucleic acid sequence with at least 70% similarity to SEQ ID NO: 3; or a first amplification oligonucleotide comprising a nucleic acid sequence with at least 70% similarity to SEQ ID NO: 4, a second amplification oligonucleotide comprising a nucleic acid sequence with at least 70% similarity to SEQ ID NO: 5, and a probe oligonucleotide comprising a nucleic acid sequence with at least 70% similarity to SEQ ID NO: 6; or a combination thereof. In some embodiments, the probe oligonucleotides may comprise a detectable label. In some embodiments, the detectable label is a fluorophore. In some embodiments, the probe oligonucleotides may comprise a quencher moiety.

The disclosure also provides methods for detecting monkeypox virus in a sample comprising: contacting a sample with the set of oligonucleotides disclosed herein and reagents for amplification; amplifying one or more target monkeypox virus nucleic acid sequences present in the sample; hybridizing one or more of the oligonucleotide probes to one or more amplified target monkeypox virus nucleic acid sequences; and detecting hybridization of the one or more probe oligonucleotide sequences to the one or more amplified monkeypox virus target nucleic acid sequences by measuring a signal from the detectable labels. In some embodiments, the sample comprises a blood product, an oropharyngeal swab, a rectal swab, a genital swab, saliva, urine, or semen. In some embodiments, the sample comprises a skin lesion specimen.

In some embodiments, the reagents for amplification comprise one or more or each of a nicking enzyme, a polymerase, a single-strand binding protein, a recombinase, a helicase, a resolvase, an enzyme cofactor, a buffer, deoxyribonucleotide or ribonucleotide triphosphates, or combinations thereof.

In some embodiments, the presence of one or more signals from the detectable label indicates hybridization of the one or more probe oligonucleotides to the one or more amplified monkeypox virus target nucleic acid sequences.

Additionally, the disclosure provides a system for detecting monkeypox virus in a sample. In some embodiments, the system comprises a set of oligonucleotides, as disclosed herein; one or more nucleic acid amplification reagents; and a nucleic acid amplification reaction system configured to amplify one or more target monkeypox virus nucleic acid sequences in the sample.

In some embodiments, the nucleic acid amplification reaction system is further configured to facilitate hybridization of one or more of the probe oligonucleotides to amplified target monkeypox virus nucleic acid sequences. In some embodiments, the nucleic acid amplification reaction system is further configured to detect hybridization of one or more of the probe oligonucleotides to amplified target monkeypox virus nucleic acid sequences. In some embodiments, the nucleic acid amplification reaction system is further configured to facilitate hybridization of one or more of the probe oligonucleotides to amplified target monkeypox virus nucleic acid sequences and detect hybridization of one or more of the probe oligonucleotides to amplified target monkeypox virus nucleic acid sequences. In some embodiments, the nucleic acid amplification reaction system is further configured to measure a signal from one or more detectable label.

In some embodiments, the system further comprises an internal control sequence, primer and probe oligonucleotide sequences for amplifying and detecting an internal control (IC) sequence, or a combination thereof.

In some embodiments, the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

In some embodiments, the sample comprises a blood product, an oropharyngeal swab, a rectal swab, a genital swab, saliva, urine, or semen. In some embodiments, the sample comprises a skin lesion specimen.

The disclosure also provides a reaction mixture comprising one or more nucleic acid amplification reagents; a set of oligonucleotides as disclosed herein; and a sample suspected of containing monkeypox virus.

In some embodiments, the reaction mixture further comprises an internal control sequence, primer and probe oligonucleotide sequences for amplifying and detecting an internal control (IC) sequence, or a combination thereof.

In some embodiments, the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

In some embodiments, the sample comprises a blood product, an oropharyngeal swab, a rectal swab, a genital swab, saliva, urine, or semen. In some embodiments, the sample comprises a skin lesion specimen.

The disclosure further provides kits for detecting monkeypox virus in a sample.

In a second embodiment, the set may comprise a first amplification oligonucleotide comprising a nucleic acid sequence with at least 70% similarity to any of SEQ ID NOs: 7-9, a second amplification oligonucleotide comprising a nucleic acid sequence with at least 70% similarity to any of SEQ ID NOs: 10-11 and a probe oligonucleotide comprising a nucleic acid sequence with at least 70% similarity to any of SEQ ID NOs: 12-15; or a first amplification oligonucleotide comprising a nucleic acid sequence with at least 70% similarity to any of SEQ ID NOs: 16-20, a second amplification oligonucleotide comprising a nucleic acid sequence with at least 70% similarity to any of SEQ ID NOs: 21-25, and a probe oligonucleotide comprising a nucleic acid sequence with at least 70% similarity to any of SEQ ID NOs: 26-39; or a combination thereof. In some embodiments, the probe oligonucleotides may comprise a detectable label. In some embodiments, the detectable label is a fluorophore. In some embodiments, the probe oligonucleotides may comprise a quencher moiety.

The disclosure also provides methods for detecting monkeypox virus in a sample comprising: contacting a sample with the set of oligonucleotides disclosed herein and reagents for amplification; amplifying one or more target monkeypox virus nucleic acid sequences present in the sample; hybridizing one or more of the oligonucleotide probes to one or more amplified target monkeypox virus nucleic acid sequences; and detecting hybridization of the one or more probe oligonucleotide sequences to the one or more amplified monkeypox virus target nucleic acid sequences by measuring a signal from the detectable labels. In some embodiments, the sample comprises a blood product, an oropharyngeal swab, a rectal swab, a genital swab, saliva, urine, or semen. In some embodiments, the sample comprises a skin lesion specimen.

In some embodiments, the reagents for amplification comprise one or more or each of a nicking enzyme, a polymerase, a single-strand binding protein, a recombinase, a helicase, a resolvase, an enzyme cofactor, a buffer, deoxyribonucleotide or ribonucleotide triphosphates, or combinations thereof.

In some embodiments, the presence of one or more signals from the detectable label indicates hybridization of the one or more probe oligonucleotides to the one or more amplified monkeypox virus target nucleic acid sequences.

Additionally, the disclosure provides a system for detecting monkeypox virus in a sample. In some embodiments, the system comprises a set of oligonucleotides, as disclosed herein; one or more nucleic acid amplification reagents; and a nucleic acid amplification reaction system configured to amplify one or more target monkeypox virus nucleic acid sequences in the sample.

In some embodiments, the nucleic acid amplification reaction system is further configured to facilitate hybridization of one or more of the probe oligonucleotides to amplified target monkeypox virus nucleic acid sequences. In some embodiments, the nucleic acid amplification reaction system is further configured to detect hybridization of one or more of the probe oligonucleotides to amplified target monkeypox virus nucleic acid sequences. In some embodiments, the nucleic acid amplification reaction system is further configured to facilitate hybridization of one or more of the probe oligonucleotides to amplified target monkeypox virus nucleic acid sequences and detect hybridization of one or more of the probe oligonucleotides to amplified target monkeypox virus nucleic acid sequences. In some embodiments, the nucleic acid amplification reaction system is further configured to measure a signal from one or more detectable label.

In some embodiments, the system further comprises an internal control sequence, primer and probe oligonucleotide sequences for amplifying and detecting one or more internal control (IC) sequences, or a combination thereof.

In some embodiments, the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

In some embodiments, the sample comprises a blood product, an oropharyngeal swab, a rectal swab, a genital swab, saliva, urine, or semen. In some embodiments, the sample comprises a skin lesion specimen.

The disclosure also provides a reaction mixture comprising one or more nucleic acid amplification reagents; a set of oligonucleotides as disclosed herein; and a sample suspected of containing monkeypox virus.

In some embodiments, the reaction mixture further comprises an internal control sequence, primer and probe oligonucleotide sequences for amplifying and detecting one or more internal control (IC) sequences, or a combination thereof.

In some embodiments, the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

In some embodiments, the sample comprises a blood product, an oropharyngeal swab, a rectal swab, a genital swab, saliva, urine, or semen. In some embodiments, the sample comprises a skin lesion specimen.

The disclosure further provides kits for detecting monkeypox virus in a sample.

Other aspects and embodiments of the disclosure will be apparent in light of the following detailed description and accompanying figures.

### BRIEF DESCRIPTIONS OF THE DRAWINGS

FIG. 1 is qPCR results for MPXV plasmid assay (left) and the internal control (right).

FIG. 2 is qPCR results for MPXV genomic DNA assay (left) and the internal control (right).

FIG. 3A is a schematic of a dual target design to detect B7R and J2R genes of the MPXV genome. FIG. 3B shows detection of MPXV and internal controls in an exemplary RT-PCR based method.

### DETAILED DESCRIPTION

The present disclosure is predicated, at least in part, on the development of a collection of oligonucleotide sequences that facilitate rapid detection of known genomic signatures for monkeypox viruses. Many current monkeypox nucleic acid assays are not specific as they cross-react with other orthopoxviral DNA. The methods described herein do not cross react with other orthopox viral DNA (e.g., variola, cowpox, camelpox, taterapox, ectromelia virus and vaccinia). Additionally, the disclosed methods employ internal controls, for example, an endogenous cellular target control and an exogenous control, which validate the assays for sample adequacy, sample extraction and amplification efficiency.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Although the terms “first,” “second,” “third,” etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first,” “second,” and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

The term “oligonucleotide,” as used herein, refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99, or 100 nucleotides, or a range defined by any of the foregoing values). The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, for example, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

Oligonucleotides can be single-stranded or double-stranded or can contain portions of both double-stranded and single-stranded sequences. The oligonucleotide can be DNA, both genomic and complimentary DNA (cDNA), RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Oligonucleotides can be obtained by chemical synthesis methods or by recombinant methods.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, which do not align with the reference sequence, are not taken into account for determining sequence identity. Methods and computer programs for alignment are well known in the art, including BLAST, Align 2, and FASTA.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 1. AMPLIFICATION AND PROBE OLIGONUCLEOTIDES

In one embodiment, the oligonucleotides described herein may be used for nucleic acid amplification (e.g., primers) or as probes for nucleic acid hybridization and detection. The terms “primer,” “primer sequence,” “primer oligonucleotide,” and “amplification oligonucleotide” as used herein, refer to an oligonucleotide which is capable of acting as a point of initiation of synthesis of an extension product that is a complementary strand of nucleic acid (all types of DNA or RNA) when placed under suitable amplification conditions (e.g., buffer, salt, temperature and pH) in the presence of nucleotides and an agent for nucleic acid polymerization (e.g., a DNA-dependent or RNA-dependent polymerase). The amplification oligonucleotides of the present disclosure can be of any suitable size, and desirably comprise, consist essentially of, or consist of about 15 to 50 nucleotides, preferably about 20 to 40 nucleotides. The oligonucleotides of the present disclosure can contain additional nucleotides in addition to those described herein.

The terms “probe,” “probe sequence,” and “probe oligonucleotide,” refer to an oligonucleotide that can selectively hybridize to at least a portion of a target sequence (e.g., a portion of a target sequence that has been amplified) under appropriate hybridization conditions. In general, a probe sequence is identified as being either “complementary” (e.g., complementary to the coding or sense strand (+)), or “reverse complementary” (e.g., complementary to the anti-sense strand (−)). The probes of the present disclosure can be of any suitable size, and desirably comprise, consist essentially of, or consist of about 10-50 nucleotides, preferably about 12-35 nucleotides.

As used herein, the terms “set,” “primer set,” “probe set,” and “primer and probe set,” refer to two or more oligonucleotides which together are capable of priming the amplification of a target sequence or target nucleic acid of interest (e.g., a target sequence within the monkeypox virus) and/or at least one probe which can detect the target sequence or target nucleic acid. In certain embodiments, the term “set” refers to a pair of oligonucleotides including a first oligonucleotide that hybridizes with the 5′-end of the target sequence or target nucleic acid to be amplified and a second oligonucleotide that hybridizes with the complement of the target sequence or target nucleic acid to be amplified.

The set of oligonucleotides described herein may be used to amplify and detect one or more target monkeypox virus sequences in a sample. The terms “target sequence” and “target nucleic acid” are used interchangeably herein and refer to a specific nucleic acid sequence, the presence or absence of which is to be detected by the disclosed method. In the context of the present disclosure, a target sequence preferably includes a nucleic acid sequence to which one or more oligonucleotides will hybridize and from which amplification will initiate. The target sequence can also include a probe-hybridizing region with which a probe may form a stable hybrid under appropriate amplification conditions. A target sequence may be single-stranded or double-stranded. The target monkeypox sequence may be within the gene encoding DNA polymerase (E9L) or envelope protein (B6R).

In some embodiments, the set comprises a first amplification oligonucleotide, a second amplification oligonucleotide, and a probe oligonucleotide. In some embodiments, the set comprises two or more first amplification oligonucleotides, two or more second amplification oligonucleotides, and two or more probe oligonucleotides.

In some embodiments, the set comprises a first amplification oligonucleotide comprising a nucleic acid sequence with at least 70% (e.g., 75%., 80%, 85%, 90%, 91%, 92%, 93%, 94%, 95%, 96%, 97%, 98%, 99%, or 100%) similarity to SEQ ID NO: 1, a second amplification oligonucleotide comprising a nucleic acid sequence with at least 70% similarity to SEQ ID NO: 2 and a probe oligonucleotide comprising a nucleic acid sequence with at least 70% similarity to SEQ ID NO: 3. In some embodiments, the set comprises a first amplification oligonucleotide comprising a nucleic acid sequence with at least 70% similarity to SEQ ID NO: 4, a second amplification oligonucleotide comprising a nucleic acid sequence with at least 70% similarity to SEQ ID NO: 5, and a probe oligonucleotide comprising a nucleic acid sequence with at least 70% similarity to SEQ ID NO: 6.

In some embodiments, the set comprises a first amplification oligonucleotide comprising a nucleic acid sequence with at least 70% similarity to SEQ ID NO: 1, a second amplification oligonucleotide comprising a nucleic acid sequence with at least 70% similarity to SEQ ID NO: 2 a probe oligonucleotide comprising a nucleic acid sequence with at least 70% similarity to SEQ ID NO: 3, a first amplification oligonucleotide comprising a nucleic acid sequence with at least 70% similarity to SEQ ID NO: 4, a second amplification oligonucleotide comprising a nucleic acid sequence with at least 70% similarity to SEQ ID NO: 5, and a probe oligonucleotide comprising a nucleic acid sequence with at least 70% similarity to SEQ ID NO: 6.

TABLE 1
Oligonucleotide Sequences

| | Sequence 5′ to 3′ | Gene |
|---|---|---|
| SEQ ID NO: 1 | CTCGTTCGTAATCCATTTCCATTAG | E9L |
| SEQ ID NO: 2 | AACGGAACAGGAAATACAAGAAG | E9L |
| SEQ ID NO: 3 | ACTGTATCCTCAAACATCCTCTATCGACGG | E9L |
| SEQ ID NO: 4 | GTCTCCATCATCCACATGTATC | B6R |
| SEQ ID NO: 5 | CCACTGGATCAAATTCTTCGTTAG | B6R |
| SEQ ID NO: 6 | TCGTACACATGTTGGGAGTATGGGATTCC | B6R |

In yet other embodiments, the set comprises a first amplification oligonucleotide comprising a nucleic acid sequence with at least 70% (e.g., 75%., 80%, 85%, 90%, 91%, 92%, 93%, 94%, 95%, 96%, 97%, 98%, 99%, or 100%) similarity to any of SEQ ID NOs: 7-9, a second amplification oligonucleotide comprising a nucleic acid sequence with at least 70% similarity to any of SEQ ID NOs: 10-11 and a probe oligonucleotide comprising a nucleic acid sequence with at least 70% similarity to any of SEQ ID NOs: 12-15; or a first amplification oligonucleotide comprising a nucleic acid sequence with at least 70% similarity to any of SEQ ID NOs: 16-20, a second amplification oligonucleotide comprising a nucleic acid sequence with at least 70% similarity to any of SEQ ID NOs: 21-25, and a probe oligonucleotide comprising a nucleic acid sequence with at least 70% similarity to any of SEQ ID NOs: 26-39; or a combination thereof.

In select embodiments, the set comprises a first amplification oligonucleotide comprising a nucleic acid sequence with at least 70% (e.g., 75%., 80%, 85%, 90%, 91%, 92%, 93%, 94%, 95%, 96%, 97%, 98%, 99%, or 100%) similarity to SEQ ID NO: 7, a second amplification oligonucleotide comprising a nucleic acid sequence with at least 70% similarity to SEQ ID NO: 10 and a probe oligonucleotide comprising a nucleic acid sequence with at least 70% similarity to SEQ ID NO: 12; or a first amplification oligonucleotide comprising a nucleic acid sequence with at least 70% similarity to SEQ ID NO: 16, a second amplification oligonucleotide comprising a nucleic acid sequence with at least 70% similarity to SEQ ID NO: 21, and a probe oligonucleotide comprising a nucleic acid sequence with at least 70% similarity to SEQ ID NO: 26.

In select embodiments, the set comprises a first amplification oligonucleotide comprising a nucleic acid sequence with at least 70% similarity to SEQ ID NO: 7. In select embodiments, the set comprises a second amplification oligonucleotide comprising a nucleic acid sequence with at least 70% similarity to SEQ ID NO: 10. In select embodiments, the set comprises a probe oligonucleotide comprising a nucleic acid sequence with at least 70% similarity to SEQ ID NO: 12.

In select embodiments, the set comprises a first amplification oligonucleotide comprising a nucleic acid sequence with at least 70% similarity to SEQ ID NO: 16. In select embodiments, the set comprises a second amplification oligonucleotide comprising a nucleic acid sequence with at least 70% similarity to SEQ ID NO: 21. In select embodiments, the set comprises a probe oligonucleotide comprising a nucleic acid sequence with at least 70% similarity to SEQ ID NO: 26.

In some embodiments, the set comprises a first amplification oligonucleotide comprising a nucleic acid sequence of any of SEQ ID NOs: 7-9, a second amplification oligonucleotide comprising a nucleic acid sequence of any of SEQ ID NOs: 10-11 and a probe oligonucleotide comprising a nucleic acid sequence of any of SEQ ID NOs: 12-15; or a first amplification oligonucleotide comprising a nucleic acid sequence of any of SEQ ID NOs: 16-20, a second amplification oligonucleotide comprising a nucleic acid sequence of any of SEQ ID NOs: 21-25, and a probe oligonucleotide comprising a nucleic acid sequence of any of SEQ ID NOs: 26-39; or a combination thereof.

TABLE 2
Oligonucleotide Sequences

| | Sequence 5′ to 3′ | Gene |
|---|---|---|
| SEQ ID NO: 7 | GTACGTACTATGCCAAATGAATC | B7R |
| SEQ ID NO: 8 | TACAGGCTATTTACAAGATGCA | B7R |
| SEQ ID NO: 9 | CACGTGTTAAACAATGGGTGATG | B7R |
| SEQ ID NO: 10 | GGTACCCACACATACAGTATC | B7R |
| SEQ ID NO: 11 | CGGTACCCACACATACAGTAT | B7R |
| SEQ ID NO: 12 | ACGTGTTAAACAATGGGTGATGGATACACT | B7R |
| SEQ ID NO: 13 | ATTCATTCATCATTATACCATTAAGT | B7R |
| SEQ ID NO: 14 | ATTCATTCATCATTATACCATTAAGTGT | B7R |
| SEQ ID NO: 15 | AGTGTATCCATCACCCATTGTTTAACACGT | B7R |
| SEQ ID NO: 16 | CAGGGTGTAGAACATGTATTTCTA | J2R |
| SEQ ID NO: 17 | AAACGGAAGAGATTTAGCACCAC | J2R |
| SEQ ID NO: 18 | TAAACGGAAGAGATTTAGCACCA | J2R |
| SEQ ID NO: 19 | TAAACGGAAGAGATATAGCACCA | J2R |
| SEQ ID NO: 20 | AAACGGAAGAGATATAGCACCAC | J2R |
| SEQ ID NO: 21 | GACGGTGTGAGAATATGTTCCGGGACCA | J2R |
| SEQ ID NO: 22 | AGTTCCCGGAGGACACGATAG | J2R |
| SEQ ID NO: 23 | AGGACACGATAGACAACATAGATTAC | J2R |
| SEQ ID NO: 24 | GACGGTGTGAGAATATGTTCCGG | J2R |
| SEQ ID NO: 25 | TGTGAGAATATGTTCCGGGACCA | J2R |
| SEQ ID NO: 26 | CAGATGACGTCTCCGGTAGAC | J2R |
| SEQ ID NO: 27 | CAGAAGCCGTAATCTATGTTGT | J2R |
| SEQ ID NO: 28 | ACAACATAGATTACGGCTTCTG | J2R |
| SEQ ID NO: 29 | CGAATACAGAAGCCGTAATCTATGTTGT | J2R |
| SEQ ID NO: 30 | CTTTACATTTTCCATTGGATGGTGCA | J2R |
| SEQ ID NO: 31 | GTCTACCGGAGACGTCATCTG | J2R |
| SEQ ID NO: 32 | CGTCTACCGGAGACGTCATCTG | J2R |
| SEQ ID NO: 33 | TCTACCGGAGACGTCATCTGTT | J2R |
| SEQ ID NO: 34 | CAGATGACGTCTCCGGTAGACG | J2R |
| SEQ ID NO: 34 | AACAGATGACGTCTCCGGTAGA | J2R |
| SEQ ID NO: 36 | GGATACACGTCTACCGGAGACG | J2R |
| SEQ ID NO: 37 | CGTCTCCGGTAGACGTGTATCC | J2R |
| SEQ ID NO: 38 | ATACACGTCTACCGGAGACG | J2R |
| SEQ ID NO: 39 | CGTCTCCGGTAGACGTGTAT | J2R |

The set of oligonucleotides for detecting monkeypox virus described above may further comprise primer and probe oligonucleotide sequences for amplifying and detecting one or more internal control (IC) sequences. The internal controls may be any suitable non-monkeypox nucleic acid sequence, including, for example, a nucleic acid sequence encoding GAPDH, beta globulin, beta-globin, beta-actin, R18, or 16S RNA. In some embodiments, the internal control may comprise an endogenous cellular control target and an exogenous control target. In one embodiment, for example, the internal control may be an endogenous human DNA sequence. In one embodiment, for example, the internal control may comprise an DNA sequence derived or obtained from the hydroxypyruvate reductase gene of the pumpkin plant, Cucurbita pepo.

Any of the oligonucleotides described herein may be modified in any suitable manner so as to stabilize or enhance the binding affinity of the oligonucleotide for its target. For example, an oligonucleotide sequence as described herein may comprise one or more modified oligonucleotide bases. Furthermore, any of the sequences listed which include internal spacers or modifications may be used without the modifications or spacers.

Any of the oligonucleotides described herein may include, for example, spacers, blocking groups, and modified nucleotides. Modified nucleotides are nucleotides or nucleotide triphosphates that differ in composition and/or structure from natural nucleotides and nucleotide triphosphates. Modifications include those naturally occurring that result from modification by enzymes that modify nucleotides, such as methyltransferases. Modified nucleotides also include synthetic or non-naturally occurring nucleotides. For example, modified nucleotides include those with 2′ modifications, such as 2′-O-methyl and 2′-fluoro. Other 2′-modified nucleotides are known in the art and are described in, for example U.S. Pat. No. 9,096,897, which is incorporated herein by reference in its entirety. Modified nucleotides or nucleotide triphosphates used herein may, for example, be modified in such a way that, when the modifications are present on one strand of a double-stranded nucleic acid where there is a restriction endonuclease recognition site, the modified nucleotide or nucleotide triphosphates protect the modified strand against cleavage by restriction enzymes.

Blocking groups or polymerase-arresting molecules are chemical moieties that inhibit target sequence-independent nucleic acid polymerization by the polymerase. The blocking group may render the oligonucleotide capable of binding a target nucleic acid molecule, but incapable of supporting template extension utilizing the detectable oligonucleotide probe as a target. For example, the presence of one or more moieties which does not allow polymerase progression likely causes polymerase arrest in non-nucleic acid backbone additions to the oligonucleotide or through stalling of a replicative polymerase. Oligonucleotides with these moieties may prevent or reduce illegitimate amplification of the probe during the course the amplification reaction. Examples of blocking groups include, for example, alkyl groups, non-nucleotide linkers, phosphorothioate, alkane-diol residues, peptide nucleic acid, and nucleotide derivatives lacking a 3′-OH, including, for example, cordycepin, spacer moieties, damaged DNA bases and the like. Examples of spacers, include, for example, C3 spacers. Spacers may be used, for example, within the oligonucleotide, and also, for example, at the ends to attach other groups, such as, for example, labels.

Any of the oligonucleotide sequences described herein may comprise, consist essentially of, or consist of a complement of any of the sequences disclosed herein. The terms “complement” or “complementary sequence,” as used herein, refer to a nucleic acid sequence that forms a stable duplex with an oligonucleotide described herein via Watson-Crick base pairing rules, and typically shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% greater identity with the disclosed oligonucleotide. Nucleic acid sequence identity can be determined using any suitable mathematical algorithm or computer software known in the art, such as, for example, CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990); Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009); Soding, Bioinformatics, 21(7): 951-960 (2005); Altschul et al., Nucleic Acids Res., 25(17): 3389-3402 (1997); and Gusfield, Algorithms on Strings, Trees and Sequences, Cambridge University Press, Cambridge UK (1997), each of which is incorporated herein by reference in its entirety).

The oligonucleotides described herein may be prepared using any suitable method, a variety of which are known in the art (see, for example, Sambrook et al., Molecular Cloning. A Laboratory Manual, 1989, 2. Supp. Ed., Cold Spring Harbour Laboratory Press: New York, N.Y.; M. A. Innis (Ed.), PCR Protocols. A Guide to Methods and Applications, Academic Press: New York, N.Y. (1990); P. Tijssen, Hybridization with Nucleic Acid Probes—Laboratory Techniques in Biochemistry and Molecular Biology (Parts I and II), Elsevier Science (1993); M. A. Innis (Ed.), PCR Strategies, Academic Press: New York, N.Y. (1995); and F. M. Ausubel (Ed.), Short Protocols in Molecular Biology, John Wiley & Sons: Secaucus, N.J. (2002); Narang et al., Meth. Enzymol., 68: 90-98 (1979); Brown et al., Meth. Enzymol., 68: 109-151 (1979); and Belousov et al., Nucleic Acids Res., 25: 3440-3444 (1997), each of which is incorporated herein by reference in its entirety). Oligonucleotide pairs also can be designed using a variety of tools, such as the Primer-BLAST tool provided by the National Center of Biotechnology Information (NCBI). Oligonucleotide synthesis may be performed on oligo synthesizers such as those commercially available from Perkin Elmer/Applied Biosystems, Inc. (Foster City, CA), DuPont (Wilmington, DE), or Milligen (Bedford, MA). Alternatively, oligonucleotides can be custom made and obtained from a variety of commercial sources well-known in the art, including, for example, the Midland Certified Reagent Company (Midland, TX), Eurofins Scientific (Louisville, KY), BioSearch Technologies, Inc. (Novato, CA), and the like. Oligonucleotides may be purified using any suitable method known in the art, such as, for example, native acrylamide gel electrophoresis, anion-exchange HPLC (see, e.g., Pearson et al., J. Chrom., 255: 137-149 (1983), incorporated herein by reference), and reverse phase HPLC (see, e.g., McFarland et al., Nucleic Acids Res., 7: 1067-1080 (1979), incorporated herein by reference).

The sequence of the oligonucleotides can be verified using any suitable sequencing method known in the art, including, but not limited to, chemical degradation (see, e.g., Maxam et al., Methods of Enzymology, 65: 499-560 (1980), incorporated herein by reference), matrix-assisted laser desorption ionization time-of-flight (MALDI-TOF) mass spectrometry (see, e.g., Pieles et al., Nucleic Acids Res., 21: 3191-3196 (1993), incorporated herein by reference), mass spectrometry following a combination of alkaline phosphatase and exonuclease digestions (Wu et al., Anal. Biochem., 290: 347-352 (2001), incorporated herein by reference), and the like.

### 2. DETECTABLE LABEL

Any one or more of the oligonucleotide sequences described herein may comprise a detectable label, such that the amplification oligonucleotide(s) and/or the probe oligonucleotide can be measured. In one embodiment, each of the probe oligonucleotide sequences described herein comprise a detectable label. The term “detectable label,” as used herein, refers to a moiety or compound that generates a signal which can be measured and whose intensity is related to (e.g., proportional to) the amount of entity bound thereto. Any suitable detectable label that can be conjugated or linked to an oligonucleotide in order to detect binding of the oligonucleotide to a target sequence can be used, many of which are known in the art. In one embodiment, the detectable label may be detected indirectly. Indirectly detectable labels are typically specific binding members used in conjunction with a “conjugate” that is attached or coupled to a directly detectable label. Coupling chemistries for synthesizing such conjugates are well-known in the art and are designed such that the specific binding property of the specific binding member and the detectable property of the label remain intact. As used herein, “specific binding member” and “conjugate” refer to the two members of a binding pair, e.g., two different molecules, where the specific binding member binds specifically to the polynucleotide of the present disclosure, and the “conjugate” specifically binds to the specific binding member. Binding between the two members of the pair is typically chemical or physical in nature. Examples of such binding pairs include, but are not limited to, antigens and antibodies, avidin/streptavidin and biotin, haptens and antibodies specific for haptens, complementary nucleotide sequences, enzyme cofactors/substrates and enzymes, and the like.

Each of the probe oligonucleotide sequences desirably comprises a detectable label. Each of the probes may be labeled with the same detectable label or different detectable labels.

In some embodiments, the detectable label may be directly detected. Such directly detectable labels include, for example, radioisotopes, fluorophores, chemiluminophores, enzymes, colloidal particles, fluorescent microparticles, intercalating dyes (e.g., SYBR Green or ethidium bromide), and the like. In select embodiments, the detectable label may be a fluorophore, such as a fluorescein-family dye, polyhalofluorescein-family dye, hexachlorofluorescein-family dye, coumarin-family dye, rhodamine-family dye, cyanine-family dye, oxazine-family dye, thiazin-family dye, squaraine-family dye, chelated lanthanide-family dye, azo-family dye, triphenylmethane-family dye, or a BODIPY®-family dye. Examples of fluorophores include, but are not limited to, FAM™ CAL-FLUOR®, QUASAR®, HEX™, JOE™, NED™, PET®, ROX™, TAMRA™, TET™, TEXAS RED®, and VIC®. One skilled in the art will appreciate that directly detectable labels may require additional components, such as substrates, triggering reagents, light, and the like, to enable detection of the label. Methods for labeling oligonucleotides, such as probes, are well-known in the art and described in, e.g., L. J. Kricka, Ann. Clin. Biochem., 39: 114-129 (2002); van Gijlswijk et al., Expert Rev. Mol. Diagn., 1: 81-91 (2001); Joos et al., J. Biotechnol., 35: 135-153 (1994); Smith et al., Nucl. Acids Res., 13: 2399-2412 (1985); Connoly et al., Nucl. Acids. Res., 13: 4485-4502 (1985); Broker et al., Nucl. Acids Res., 5: 363-384 (1978); Bayer et al., Methods of Biochem. Analysis, 26: 1-45 (1980); Langer et al., Proc. Natl. Acad. Sci. USA, 78: 6633-6637 (1981); Richardson et al., Nucl. Acids Res., 11: 6167-6184 (1983); Brigati et al., Virol., 126: 32-50 (1983); Tchen et al., Proc. Natl. Acad. Sci. USA, 81: 3466-3470 (1984); Landegent et al., Exp. Cell Res., 15: 61-72 (1984); A. H. Hopman et al., Exp. Cell Res., 169: 357-368 (1987); and Temsamani et al., Mol. Biotechnol., 5: 223-232 (1996), each of which is incorporated herein by reference in its entirety.

The fluorescent dye may be conjugated to either end of the probe oligonucleotide, or to an internal site (e.g., within the oligonucleotide sequence). In some embodiments, the probe oligonucleotide sequence comprises a fluorescent dye at the 5′ end of the oligonucleotide. In some embodiments, the probe oligonucleotide sequence comprises a fluorescein-based dye at the 5′ end of the oligonucleotide. In some embodiments, the probe oligonucleotide sequence comprises a fluorescent dye at the 3′ end of the oligonucleotide. In some embodiments, the probe oligonucleotide sequence comprises a fluorescein-based dye at the 3′ end of the oligonucleotide.

In some embodiments, any one or more of the oligonucleotides described herein may also comprise a quencher moiety. A quencher moiety is any molecule or part of a molecule that decreases the light emitted by the detectable label (e.g., fluorophore) at the wavelength at which signal is measured or serves to shift the wavelength of light emitted by the detectable label (e.g., fluorophore). For example, when a detectable label (e.g., a fluorophore) and quencher moiety are held in close proximity, such as at the ends of a probe, the quencher moiety prevents detection of a signal (e.g., fluorescence) from the detectable label. When the two moieties are physically separated, the signal becomes detectable. The quencher may be selected from any suitable quencher known in the art, such as, for example, BLACK HOLE QUENCHER® 1 (BHQ-1®), BLACK HOLE QUENCHER® 2 (BHQ-2®), IOWA BLACK® FQ, and IOWA BLACK® RQ. For example, the oligonucleotide probe may comprise a FAM fluorophore, CAL-FLUOR®, or QUASAR fluorophore and a BHQ-1 or BHQ-2 quencher.

The quencher moiety may be conjugated to either end of the probe oligonucleotide, or to an internal site (e.g., within the oligonucleotide sequence). In some embodiments, the probe oligonucleotide sequence comprises a quencher moiety at the 3′ end of the oligonucleotide. In some embodiments, the probe oligonucleotide sequence comprises a quencher moiety at the 5′ end of the oligonucleotide. Preferably, the quencher moiety is distant from the detectable label (e.g., at the opposite end of the oligonucleotide).

The internal control probe also desirably comprises a detectable label. In some embodiments, the internal control probe may comprise a different label than the probes used to detect monkeypox virus, which allows for simultaneous detection and differentiation of internal control and monkeypox virus-amplified products within the same reaction. The internal control probe may also comprise a quencher moiety.

The selection of a particular label and labeling technique will depend on several factors, such as the ease and cost of the labeling method, spectral spacing between different detectable labels used, the quality of sample labeling desired, the effects of the detectable moiety on the hybridization reaction (e.g., on the rate and/or efficiency of the hybridization process), the nature of the amplification method used, the nature of the detection system, the nature and intensity of the signal generated by the detectable label, and the like.

### 3. METHODS FOR AMPLIFYING AND DETECTING MONKEYPOX

The present disclosure provides methods for detecting monkeypox in a sample. The methods comprise one or more of: contacting a sample with one or more sets of oligonucleotides disclosed herein and reagents for amplification; amplifying one or more target monkeypox virus nucleic acid sequences present in the sample; hybridizing one or more of the oligonucleotide probes to one or more amplified target monkeypox virus nucleic acid sequences; and detecting hybridization of the one or more probe oligonucleotide sequences to the one or more amplified monkeypox target nucleic acid sequences by measuring a signal from the detectable labels. Descriptions of the oligonucleotides set forth herein with respect to the aforementioned set of oligonucleotides also are applicable to the disclosed method.

The sample can be any suitable sample obtained from any suitable subject, typically a mammal (e.g., squirrels, mice, rats, non-human primates, or humans). Preferably, the subject is a human. The sample may be obtained from any suitable biological source, such as, a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. In some embodiments, the sample is obtained from a blood product (e.g., whole blood, serum, plasma, or other blood source), an oropharyngeal swab, a rectal swab, a genital swab, saliva, urine, or semen.

In some embodiments, the sample is a skin lesion specimen (e.g., fluid, tissue, or other material from a monkeypox lesion). Skin lesion specimens, include but are not limited to swabs of lesion surface and/or lesion fluid or exudate, roofs from one or more lesions, or lesion crusts. In some embodiments, the lesion specimen is a lesion swab.

The sample can be obtained from the subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like. The sample may be refrigerated (2-8 °C) or frozen (−20 °C or lower) following collection and prior to analysis.

After the sample is obtained from a subject, the sample may be contacted with the set of oligonucleotides, comprising amplification oligonucleotides and probes as described herein, and amplification reagents to form a reaction mixture. In some embodiments, primer and probe oligonucleotide sequences for amplifying and detecting an internal control (IC) sequence(s), as described herein, are added to each sample. The internal control(s) are then processed through the entire sample preparation and amplification procedure along with the test samples, to demonstrate proper sample processing and assay validity. Alternatively, internal control primer and probe oligonucleotide sequences may be added to the reaction mixture after sample processing.

In some embodiments, the set of oligonucleotides useful for amplification may comprise a first amplification oligonucleotide comprising a nucleic acid sequence with at least 70% similarity to SEQ ID NO: 1, a second amplification oligonucleotide comprising a nucleic acid sequence with at least 70% similarity to SEQ ID NO: 2, and a probe oligonucleotide comprising a nucleic acid sequence with at least 70% similarity to SEQ ID NO: 3 and/or a first amplification oligonucleotide comprising a nucleic acid sequence with at least 70% similarity to SEQ ID NO: 4, a second amplification oligonucleotide comprising a nucleic acid sequence with at least 70% similarity to SEQ ID NO: 5, and a probe oligonucleotide comprising a nucleic acid sequence with at least 70% similarity to SEQ ID NO: 6.

In yet other embodiments, the set of oligonucleotides useful for amplification may comprise a first amplification oligonucleotide comprising a nucleic acid sequence with at least 70% (e.g., 75%., 80%, 85%, 90%, 91%, 92%, 93%, 94%, 95%, 96%, 97%, 98%, 99%, or 100%) similarity to any of SEQ ID NOs: 7-9, a second amplification oligonucleotide comprising a nucleic acid sequence with at least 70% similarity to any of SEQ ID NOs: 10-11 and a probe oligonucleotide comprising a nucleic acid sequence with at least 70% similarity to any of SEQ ID NOs: 12-15; or a first amplification oligonucleotide comprising a nucleic acid sequence with at least 70% similarity to any of SEQ ID NOs: 16-20, a second amplification oligonucleotide comprising a nucleic acid sequence with at least 70% similarity to any of SEQ ID NOs: 21-25, and a probe oligonucleotide comprising a nucleic acid sequence with at least 70% similarity to any of SEQ ID NOs: 26-39; or a combination thereof.

In select embodiments, the set comprises a first amplification oligonucleotide comprising a nucleic acid sequence with at least 70% similarity to SEQ ID NO: 7. In select embodiments, the set comprises a second amplification oligonucleotide comprising a nucleic acid sequence with at least 70% similarity to SEQ ID NO: 10. In select embodiments, the set comprises a probe oligonucleotide comprising a nucleic acid sequence with at least 70% similarity to SEQ ID NO: 12.

In select embodiments, the set comprises a first amplification oligonucleotide comprising a nucleic acid sequence with at least 70% similarity to SEQ ID NO: 16. In select embodiments, the set comprises a second amplification oligonucleotide comprising a nucleic acid sequence with at least 70% similarity to SEQ ID NO: 21. In select embodiments, the set comprises a probe oligonucleotide comprising a nucleic acid sequence with at least 70% similarity to SEQ ID NO: 26.

In some embodiments, the set comprises a first amplification oligonucleotide comprising a nucleic acid sequence of any of SEQ ID NOs: 7-9, a second amplification oligonucleotide comprising a nucleic acid sequence of any of SEQ ID NOs: 10-11 and a probe oligonucleotide comprising a nucleic acid sequence of any of SEQ ID NOs: 12-15; or a first amplification oligonucleotide comprising a nucleic acid sequence of any of SEQ ID NOs: 16-20, a second amplification oligonucleotide comprising a nucleic acid sequence of any of SEQ ID NOs: 21-25, and a probe oligonucleotide comprising a nucleic acid sequence of any of SEQ ID NOs: 26-39; or a combination thereof.

The reaction mixture is then placed under amplification conditions. The term “amplification conditions,” as used herein, refers to conditions that promote annealing and/or extension of the amplification oligonucleotides. Such conditions are well-known in the art and depend on the selected amplification method. For example, PCR amplification conditions generally comprise thermal cycling, e.g., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and/or temperature cycling, buffer, salt, ionic strength, pH, and the like.

As used herein, the phrase “amplification reagents” refers to reagents used in amplification reactions and may include, but is not limited to, buffers, reagents, enzymes having reverse transcriptase and/or polymerase activity or exonuclease activity; enzyme cofactors such as magnesium or manganese; salts; and deoxynucleotide triphosphates (dNTPs) such as deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP). Particular amplification reagents, and combinations thereof, are dependent on the amplification method and conditions employed.

Amplifying a monkeypox virus nucleic acid sequence in the sample can be performed using any suitable nucleic acid sequence amplification method known in the art. In some embodiments, the amplification includes, but is not limited to, polymerase chain reaction (PCR), reverse-transcriptase PCR (RT-PCR), real-time PCR, digital PCR, transcription-mediated amplification (TMA), rolling circle amplification, nucleic acid sequence-based amplification (NASBA), self-sustained sequence replication (3SR), strand displacement amplification (SDA), transcription-mediated amplification (TMA), single primer isothermal amplification (SPIA), helicase-dependent amplification (HDA), loop mediated amplification (LAMP), recombinase-polymerase amplification (RPA), and ligase chain reaction (LCR).

In some embodiments, amplification of monkeypox virus nucleic acid sequences is performed using PCR. Any suitable PCR methodology, combination of PCR methodologies, or combination of PCR with other amplification techniques may be used. Exemplary PCR methodologies include, but are not limited to, digital PCR (dPCR), competitive dPCR (cdPCR), dPCR LATE (linear-after-the-exponential), allele-specific PCR, assembly PCR, asymmetric PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, and universal fast walking PCR.

In some embodiments, amplification of monkeypox virus nucleic acid sequences is performed using real-time PCR. “Real-time PCR,” as used herein, refers to a PCR method in which the accumulation of amplification product is measured as the reaction progresses, in real time, with product quantification after each cycle, in contrast to conventional PCR in which the amplified DNA product is detected in an end-point analysis. Real-time PCR also is known in the art as quantitative PCR (qPCR). Real-time detection of PCR products typically involves the use of non-specific fluorescent dyes that intercalate with any double-stranded DNA and sequence-specific fluorescently-labeled DNA probes. Real-time PCR techniques and systems are known in the art (see, e.g., Dorak, M. Tevfik, ed. Real-time PCR. Taylor & Francis (2007); and Fraga et al., Current protocols essential laboratory techniques: 10-3 (2008), each of which is incorporated herein by reference in its entirety) and are commercially available from a variety of sources (e.g., m2000rt REALTIME™ PCR system (Abbott Molecular, Inc., Des Plaines, IL); CFX Real-Time PCR Detection Systems (Bio-Rad Laboratories, Inc., Hercules, CA); and TAQMAN™ Real-Time PCR System (ThermoFisher Scientific, Waltham, MA)), any of which can be employed in the methods described herein.

The amplification may be performed using isothermal amplification.

Strand-displacement amplification combines the ability of a restriction endonuclease to nick the unmodified strand of its target DNA and the action of an exonuclease-deficient DNA polymerase to extend the 3′ end at the nick and displace the downstream DNA strand at a fixed temperature (See, Walker et al., Proc. Natl. Acad. Sci. USA, 1992). Primers used in SDA include a restriction endonuclease recognition at site 5′ to the target binding sequence (See, U.S. Pat. Nos. 5,270,184 and 5,344,166, each of which is incorporated herein by reference).

Nucleic acid sequence based amplification (NASBA) uses three enzymes (e.g., RNase H, avian myeloblastosis virus (AMV) reverse transcriptase and T7 RNA polymerase) working in concert at a low isothermal temperature, generally 41 °C. (See, Compton, Nature, 1991, 350:91-92; Chan et al., Rev. Med. Microbiol., 1999, 10: 185-196). The self-sustained sequence replication (3SR) reaction is a very efficient method for isothermal amplification of target DNA or RNA sequences. A 3SR system involves the collective activities of AMV reverse transcriptase, E. coli RNase H, and DNA-dependent RNA polymerase (e.g., T7 RNA polymerase). Transcription-mediated amplification (TMA) uses an RNA polymerase to make RNA from a promoter engineered in the primer region, a reverse transcriptase to produce complementary DNA from the RNA templates and RNase H to remove the RNA from cDNA (See, Guatelli et al., Proc. Natl. Acad. Sci. USA, 1990).

NASBA, 3SR, and TMA primers comprise an RNA polymerase promoter linked to the target binding sequence of the primer. Promoters or promoter sequences for incorporation in the primers are nucleic acid sequences (either naturally occurring, produced synthetically or a product of a restriction digest) that are specifically recognized by an RNA polymerase that recognizes and binds to that sequence and initiates the process of transcription whereby RNA transcripts are generated. Examples of useful promoters include those which are recognized by certain bacteriophage polymerases such as those from bacteriophage T3, T7 or SP6 or a promoter from E. coli.

The isothermal amplification methods may rely on nicking and extension reactions, “nicking and extension amplification,” to amplify shorter sequences in a quicker timeframe than traditional amplification reactions. These methods may include, for example, reactions that use only two amplification oligonucleotides, one or two nicking enzymes, and a polymerase, under isothermal conditions.

In nicking and extension amplification, a target nucleic acid sequence, having a sense and antisense strand, is contacted with a pair of amplification oligonucleotides. The first amplification oligonucleotide comprises a nucleic acid sequence comprising a recognition region at the 3′ end that is complementary to the 3′ end of the target sequence antisense strand, a nicking enzyme site upstream of said recognition region, and a stabilizing region upstream of said nicking enzyme site. The second amplification oligonucleotide comprises a nucleotide sequence comprising a recognition region at the 3′ end that is complementary to the 3′ end of the target sequence sense strand, a nicking enzyme site upstream of said recognition region, and a stabilizing region upstream of said nicking enzyme site. Two nicking enzymes are provided. One nicking enzyme is capable of nicking at the nicking enzyme site of the first amplification oligonucleotide but incapable of nicking within said target sequence. The other nicking enzyme is capable of nicking at the nicking enzyme site of the second amplification oligonucleotide but incapable of nicking within said target sequence. A DNA polymerase is employed under conditions for amplification which involves multiple cycles of extension of the amplification oligonucleotides thereby producing a double-stranded nicking enzyme site which are nicked by the nicking enzymes to produce the amplification product. For example, see U.S. Pat. Nos. 9,689,031; 9,617,586; 9,562,264; and 9,562,263, and U.S. patent application Ser. Nos. 15/467,893; 15/600,951; and 16/243,829, each of which is incorporated herein by reference in its entirety.

Recombinase-polymerase amplification (RPA) relies on the properties of recombinase and related proteins, to invade double-stranded DNA with single stranded homologous DNA permitting sequence specific priming of DNA polymerase reactions. In RPA, a recombinase agent is contacted with first and second amplification oligonucleotides to form nucleoproteins. These nucleoproteins contact the target sequence to form a first double stranded structure at a first portion of said first strand and form a double stranded structure at a second portion of said second strand so the 3′ ends of said first amplification oligonucleotide and the second amplification oligonucleotide are oriented towards each other on the DNA comprising the target sequence. The 3′ end of the amplification oligonucleotides in the nucleoprotein are extended by DNA polymerases to generate first and second double stranded nucleic acids, and first and second displaced strands of nucleic acid. The steps are repeated until the desired level of amplification is achieved. Methods and materials useful for RPA of a target nucleic acid sequence are known in the art. For example, see U.S. Pat. Nos. 7,270,981; 8,460,875; 7,399,590; 7,666,598; 8,030,000; 8,426,134; 8,945,845; 9,663,820; 10,329,603; 10,329,602; 8,017,339; 8,574,846; 8,962,255; 10,036,057; 8,071,308; 10,093,908; 8,637,253; 10,947,584; 11,339,382, and U.S. Patent Publication No. 2017/0044598 and 2015/0240298; each of which is incorporated herein by reference in its entirety.

Loop-mediated isothermal amplification (LAMP) uses 4-6 primers recognizing multiple distinct regions of target DNA for a highly specific amplification reaction. A strand-displacing DNA polymerase initiates synthesis and two specially designed primers form “loop” structures to facilitate subsequent rounds of amplification through extension on the loops and additional annealing of primers.

Ligation chain reactions (LCR) are similar to PCR with the major distinguishing feature that, in LCR, ligation instead of polymerization is used to amplify target sequences. LCR is described inter alia in Backman et al., European Patent 320 308; Landegren et al., Science 241:1077 (1988); Wu et al., Genomics 4:560 (1989). In some advanced forms of LCR, specificity can be increased by providing a gap between the oligonucleotides, which gaps must be filled in by template-dependent polymerization. Helicase-dependent amplification (HDA) utilizes a DNA helicase to generate single-stranded templates for primer hybridization and subsequent primer extension by a DNA polymerase.

Rolling circle amplification (RCA) is described by Lisby, Mol. Biotechnol. 12(1):75-99 (1999)), Hatch et al., Genet. Anal. 15(2):35-40 (1999). Rolling circle replication (RCR) is a process of unidirectional nucleic acid replication that can rapidly synthesize multiple copies of circular molecules of DNA or RNA. Two primers can be used with a small (less than 150 hp) circularized padlock DNA probe to produce hyperbranched amplification of the entire small circle (Lizardi, et al., Nature Genetics 19:225-232 (1998), Zhang, et al., Gene 211:277-285 (1998)).

Following amplification of one or more monkeypox virus nucleic acid sequences present in the sample, the disclosed methods may further comprise hybridizing one or more of the probe oligonucleotide sequences disclosed herein to the one or more amplified target monkeypox virus nucleic acid sequences.

Following hybridization of the one or more of the probe oligonucleotide sequences to the one or more amplified target nucleic acid sequences, the methods comprise detecting hybridization of the one or more probe oligonucleotide sequences to the one or more amplified target nucleic acid sequences by assessing a signal from each of the detectable labels, whereby (i) the presence of one or more signals indicates hybridization of the one or more probe oligonucleotide sequences to the one or more target monkeypox virus nucleic acid sequences and the presence of monkeypox virus in the sample, and (ii) the absence of signals indicates the absence of monkeypox virus in the sample. Detection of signals from the one or more probe oligonucleotide sequences may be performed using a variety of well-known methodologies, depending on the type of detectable label.

In certain embodiments of the present invention, the probes described herein are used to detect amplification products generated by the amplification reaction. The probes described herein can be employed using a variety of well-known homogeneous or heterogeneous methodologies.

Homogeneous detection methods include, but are not limited to, the use of FRET labels that are attached to the probes and that emit a signal in the presence of the target sequence, Molecular Beacons (See, Tyagi et al., Nature Biotechnol., 1996, 14: 303-308; Tyagi et al., Nature Biotechnol., 1998, 16: 49-53; Kostrikis et al., Science, 1998, 279: 1228-1229; Sokol et al., Proc. Natl. Acad. Sci. USA, 1998, 95: 11538-11543; Marras et al., Genet. Anal., 1999, 14: 151-156; and U.S. Pat. Nos. 5,846,726, 5,925,517, 6,277,581 and 6,235,504), and the TaqMan® assays (See, U.S. Pat. Nos. 5,210,015; 5,804,375; 5,487,792 and 6,214,979 and WO 01/86001). Using these detection techniques, products of the amplification reaction can be detected as they are formed, namely, in a real time manner. As a result, amplification product/probe hybrids are formed and detected while the reaction mixture is under amplification conditions.

Other examples of homogeneous detection methods including hybridization protection assays (HPA). In such assays, the probes are labeled with acridinium ester (AE), a highly chemiluminescent molecule (See, Weeks et al., Clin. Chem., 1983, 29: 1474-1479; Berry et al., Clin. Chem., 1988, 34: 2087-2090), using a non-nucleotide-based linker arm chemistry (See, U.S. Pat. Nos. 5,585,481 and 5,185,439). Chemiluminescence is triggered by AE hydrolysis with alkaline hydrogen peroxide, which yields an excited N-methyl acridone that subsequently deactivates with emission of a photon. In the absence of a target sequence, AE hydrolysis is rapid. However, the rate of AE hydrolysis is greatly reduced when the probe is bound to the target sequence. Thus, hybridized and un-hybridized AE-labeled probes can be detected directly in solution without the need for physical separation.

Heterogeneous detection systems are also well-known in the art and generally employ a capture agent to separate amplified sequences from other materials in the reaction mixture. Capture agents typically comprise a solid support material (e.g., microtiter wells, beads, chips, and the like) coated with one or more specific binding sequences. A binding sequence may be complementary to a tail sequence added to oligonucleotide probes of the invention. Alternatively, a binding sequence may be complementary to a sequence of a capture oligonucleotide, itself comprising a sequence complementary to a tail sequence of a probe. After separation of the amplification product/probe hybrids bound to the capture agents from the remaining reaction mixture, the amplification product/probe hybrids can be detected using any detection methods, such as those described herein.

A subject identified according to the methods described above as having monkeypox may be treated and/or monitored using routine techniques known in the art. Thus, in some embodiments, the methods further comprise: monitoring the subject for monkeypox virus infection symptoms, treating the subject for monkeypox virus infection, or monitoring the subject for monkeypox virus infection symptoms prior to or after treating the subject for monkeypox virus infection.

Treatment for monkeypox may include symptom management (e.g., use of pain relievers, fever reducers, oatmeal baths, rest, hydration) and antiviral agents, such as tecovirimat (TPOXX), brincidofovir (Tembexa) and cidofovir (Vistide), particularly for those who are at risk of severe illness (e.g., children, pregnant women, and those individuals with compromised immune systems). Treatment may also include antibiotics to treat secondary bacterial infections, if observed. A subject may be monitored prior to or after being treated for monkeypox virus infection. Such monitoring involves observing symptoms, or changes in symptoms, including presence of fever, chills, swelling of the lymph nodes, and development or exacerbations of skin rashes.

### 4. SYSTEMS

The disclosure also provides systems for amplifying and detecting monkeypox virus in a sample. In some embodiments, the system comprises a set of oligonucleotide sequences described herein. In some embodiments, the system comprises a first amplification oligonucleotide comprising a nucleic acid sequence with at least 70% similarity to SEQ ID NO: 1, a second amplification oligonucleotide comprising a nucleic acid sequence with at least 70% similarity to SEQ ID NO: 2 and a probe oligonucleotide comprising a nucleic acid sequence with at least 70% similarity to SEQ ID NO: 3. In some embodiments, the system comprises a first amplification oligonucleotide comprising a nucleic acid sequence with at least 70% similarity to SEQ ID NO: 4, a second amplification oligonucleotide comprising a nucleic acid sequence with at least 70% similarity to SEQ ID NO: 5, and a probe oligonucleotide comprising a nucleic acid sequence with at least 70% similarity to SEQ ID NO: 6.

In some embodiments, the system comprises a first amplification oligonucleotide comprising a nucleic acid sequence with at least 70% similarity to SEQ ID NO: 1, a second amplification oligonucleotide comprising a nucleic acid sequence with at least 70% similarity to SEQ ID NO: 2 a probe oligonucleotide comprising a nucleic acid sequence with at least 70% similarity to SEQ ID NO: 3, a first amplification oligonucleotide comprising a nucleic acid sequence with at least 70% similarity to SEQ ID NO: 4, a second amplification oligonucleotide comprising a nucleic acid sequence with at least 70% similarity to SEQ ID NO: 5, and a probe oligonucleotide comprising a nucleic acid sequence with at least 70% similarity to SEQ ID NO: 6.

In still yet other embodiments, the system comprises a first amplification oligonucleotide comprising a nucleic acid sequence with at least 70% (e.g., 75%., 80%, 85%, 90%, 91%, 92%, 93%, 94%, 95%, 96%, 97%, 98%, 99%, or 100%) similarity to any of SEQ ID NOs: 7-9, a second amplification oligonucleotide comprising a nucleic acid sequence with at least 70% similarity to any of SEQ ID NOs: 10-11 and a probe oligonucleotide comprising a nucleic acid sequence with at least 70% similarity to any of SEQ ID NOs: 12-15; or a first amplification oligonucleotide comprising a nucleic acid sequence with at least 70% similarity to any of SEQ ID NOs: 16-20, a second amplification oligonucleotide comprising a nucleic acid sequence with at least 70% similarity to any of SEQ ID NOs: 21-25, and a probe oligonucleotide comprising a nucleic acid sequence with at least 70% similarity to any of SEQ ID NOs: 26-39; or a combination thereof.

In select embodiments, the system comprises a first amplification oligonucleotide comprising a nucleic acid sequence with at least 70% similarity to SEQ ID NO: 7. In select embodiments, the system comprises a second amplification oligonucleotide comprising a nucleic acid sequence with at least 70% similarity to SEQ ID NO: 10. In select embodiments, the system comprises a probe oligonucleotide comprising a nucleic acid sequence with at least 70% similarity to SEQ ID NO: 12.

In select embodiments, the system comprises a first amplification oligonucleotide comprising a nucleic acid sequence with at least 70% similarity to SEQ ID NO: 16. In select embodiments, the system comprises a second amplification oligonucleotide comprising a nucleic acid sequence with at least 70% similarity to SEQ ID NO: 21. In select embodiments, the system comprises a probe oligonucleotide comprising a nucleic acid sequence with at least 70% similarity to SEQ ID NO: 26.

In some embodiments, the system comprises a first amplification oligonucleotide comprising a nucleic acid sequence of any of SEQ ID NOs: 7-9, a second amplification oligonucleotide comprising a nucleic acid sequence of any of SEQ ID NOs: 10-11 and a probe oligonucleotide comprising a nucleic acid sequence of any of SEQ ID NOs: 12-15; or a first amplification oligonucleotide comprising a nucleic acid sequence of any of SEQ ID NOs: 16-20, a second amplification oligonucleotide comprising a nucleic acid sequence of any of SEQ ID NOs: 21-25, and a probe oligonucleotide comprising a nucleic acid sequence of any of SEQ ID NOs: 26-39; or a combination thereof.

Descriptions of the oligonucleotides and sets of oligonucleotides set forth herein are applicable to those same aspects of the system described herein.

The system may further comprise one or more nucleic acid amplification reagents. Many such reagents are described herein or otherwise known in the art and commercially available. Examples of suitable reagents for inclusion in the kit (in addition to the oligonucleotides described herein) include conventional reagents employed in nucleic acid amplification reactions, such as, for example, one or more enzymes having polymerase activity, enzyme cofactors (such as magnesium or nicotinamide adenine dinucleotide (NAD)), salts, buffers, deoxyribonucleotide, or ribonucleotide triphosphates (dNTPs/rNTPs; for example, deoxyadenosine triphosphate, deoxyguanosine triphosphate, deoxycytidine triphosphate, and deoxythymidine triphosphate) blocking agents, labeling agents, and the like. Other reagents used in amplification reactions include nicking enzymes, single-strand binding proteins, helicases, resolvases, and the like. In some embodiments, the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

The system may further comprise a nucleic acid amplification reaction system configured to amplify one or more target monkeypox virus nucleic acid sequences in the sample. The nucleic acid amplification reaction system may further be configured to facilitate hybridization of one or more of the probe oligonucleotides to amplified target monkeypox virus nucleic acid sequences and/or detect hybridization of one or more of the probe oligonucleotides to amplified target monkeypox virus nucleic acid sequences. In some embodiments, the nucleic acid amplification reaction system is further configured to measure a signal from one or more detectable label.

The nucleic acid amplification reaction system may comprise a single component which is configured to carry out the above listed roles. Alternatively, the nucleic acid amplification reaction system may comprise multiple components, which in combinations are capable of carrying out the above listed roles. For example, the nucleic acid amplification reaction system may comprise one or more of: a thermocycler, an incubator or incubation chamber, a fluorometer, a spectrophotometer, a colorimeter, and the like.

### 5. KITS

The disclosure also provides a kit for amplifying and detecting monkeypox virus in a sample. The kit comprises at least one oligonucleotide as described herein. In some embodiments, the kit comprises a set of oligonucleotide sequences described herein. The kit may further comprise reagents for amplifying and detecting nucleic acid sequences, and instructions for amplifying and detecting monkeypox virus. Descriptions of the oligonucleotides and sets of oligonucleotides set forth herein with respect to the aforementioned methods also are applicable to those same aspects of the kit described herein. Many such reagents are described herein or otherwise known in the art and commercially available. Examples of suitable reagents for inclusion in the kit (in addition to the oligonucleotides described herein) include conventional reagents employed in nucleic acid amplification reactions, such as, for example, one or more enzymes having polymerase activity, enzyme cofactors (such as magnesium or nicotinamide adenine dinucleotide (NAD)), salts, buffers, deoxyribonucleotide, or ribonucleotide triphosphates (dNTPs/rNTPs; for example, deoxyadenosine triphosphate, deoxyguanosine triphosphate, deoxycytidine triphosphate, and deoxythymidine triphosphate) blocking agents, labeling agents, and the like. Other reagents used in amplification reactions include nicking enzymes, single-strand binding proteins, helicases, resolvases, and the like.

The kit may comprise instructions for using the amplification reagents and oligonucleotides described herein, e.g., for processing the test sample, extracting nucleic acid molecules, and/or performing the test; and for interpreting the results obtained. The instructions may be printed or provided electronically (e.g., DVD, CD, or available for viewing or acquiring via internet resources).

The kit may be supplied in a solid (e.g., lyophilized) or liquid form. The various components of the kit of the present disclosure may optionally be contained within different containers (e.g., vial, ampoule, test tube, flask, or bottle) for each individual component (e.g., amplification oligonucleotides, probe oligonucleotides, or buffer). Each component will generally be suitable as aliquoted in its respective container or provided in a concentrated form. Other containers suitable for conducting certain steps of the amplification/detection assay may also be provided. The individual containers are preferably maintained in close confinement for commercial sale.

### 6. EXAMPLES

### Example 1

Inclusivity: To evaluate the conservation of oligos designed to detect monkeypox or VZV targets in prototype assays, a sequence analysis pipeline was developed. First, all available monkeypox complete genome sequences from the recent outbreak were downloaded from GISAID and added to all Genbank monkeypox complete genomes under taxon ID 10244, which includes both Central and Western African lineages (date of accession Jul. 15, 2022). Likewise, all available complete genomes for VZV were downloaded from Genbank (date of accession Jun. 27, 2022). The BLAST aligner was utilized in a python pipeline to produce alignments with each appropriate oligo and complete percent identity was calculated from the local percent identity.

Exclusivity: To evaluate the potential cross reactivity of all monkeypox oligo sets with off target pathogens, an exclusivity analysis was conducted using BLAST. First, sequences were downloaded from Genbank for each taxon ID and subnodes (date of accession Jun. 29, 2022) to assemble a database that was segregated to 28 individual servers for parallel BLAST alignment processing. Duplicate sequences were removed before BLAST alignment was conducted. Results were annotated by cross-indexing to the oligo table and the group taxonomy name. Results were sorted by bit scores and the corrected percent identity was calculated by normalizing the length of the alignment to the length of the query oligo. Likewise, the total number of mismatches was corrected to account for end positions that did not align. The resulting metadata and BLAST results were then processed to generate the ‘midline’ result—the position dots (match) and dashes (mismatch), resulting in >2.4 million records. Hits were then parsed to unique oligo target accession values to remove hits that were in the same taxon ID as the intended target. Next, the individual hit with the highest corrected percent identity for each unique oligo/taxon group combination was collected. Results with 79% identity or higher were selected for further manual examination. For each oligo set, the individual oligos with 80% identity or higher were entered into a summary table (see appendix summary tables file). If an organism is not listed in the summary table for an oligo set, it is because there were no hits for any oligos with >80% identity. Hits where an amplicon was not possible due to only a single oligo having >80% identity were discarded, as well as hits where a forward and reverse oligo had >80% identity but the probe had <80%. Hits where a forward, reverse, and probe all had >80% identity were further examined for a mismatch position in the 3 nucleotides on the 3′ end, for forward and reverse primer positions on the same strand, distance between the forward and reverse oligos, and inclusion of probe in between forward and reverse primers in an investigation table (see appendix investigation tables file). Hits in which the forward and reverse oligo were on the same strand, the distance between the forward and reverse oligo were >1000 nucleotides, an insertion or deletion was identified in the alignment, or the probe was not positioned between the forward and reverse primer were discarded.

B6R and E9L oligo sets had >90% sequence identity to >99% of all monkeypox sequences analyzed, which included both Central and West African genomes. B6R oligos were predicted to detect all Central and Western African monkeypox infections. Orthopoxviruses (vaccinia, akhmeta virus, camelpox, cowpox, horsepox, rabbitpox, and taterapox) may have cross reactivity. No cross reactivity is predicted outside of the orthopoxvirus genus. E9L oligos were predicted to detect all Central and Western African monkeypox infections. Orthopoxviruses (variola, vaccinia, akhmeta virus, camelpox, ectromelia virus, horsepox, abatino virus, rabbitpox, raccoonpox, skunkpox, and taterapox) may have cross reactivity. No cross reactivity is predicted outside of the orthopoxvirus genus.

### Example 2

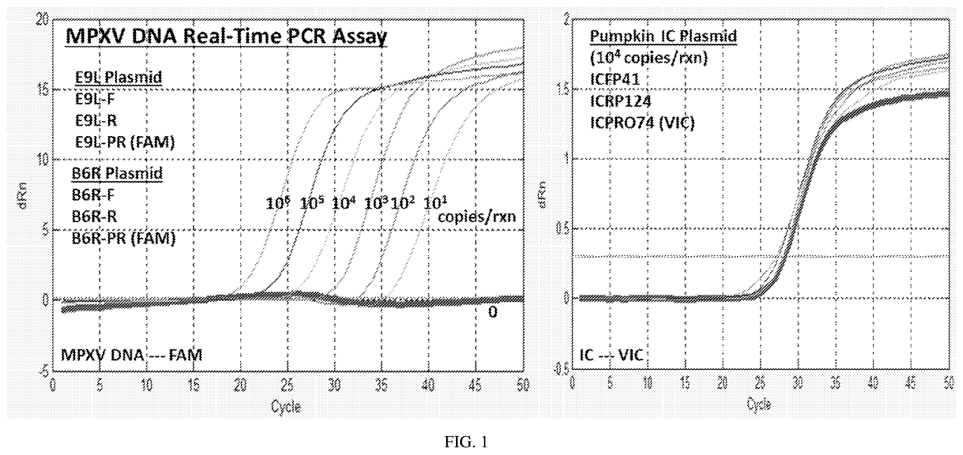

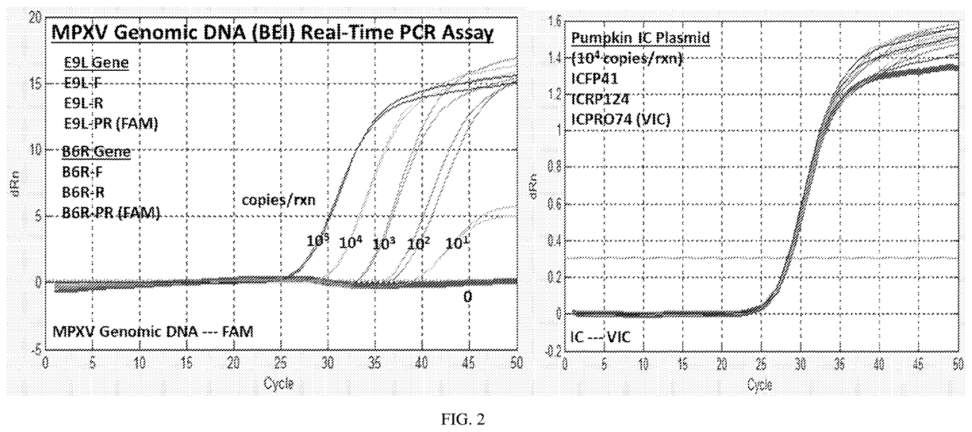

Amplification of monkeypox virus nucleic acid sequences was performed by real-time PCR using the described amplification and probe oligonucleotides. The monkeypox virus sample was provided in the reaction as plasmids carrying either E9L or B6R gene (FIG. 1) or monkeypox (MPXV) genomic DNA (FIG. 2).

### PCR Cycling Parameters:

| | Number | | | |
|---|---|---|---|---|
| | of cycles | Temperature | Time | |
| Denaturation/Activation | 1 | 95 °C | 10 | min |
| Amplification/Detection | 50 | 95 °C | 25 | s |
| | | 65 °C | 15 | s |
| | | 60 °C | 60 | s |

The PCR reaction contained equal amounts of each of the amplification and probe oligonucleotides (SEQ ID NOs: 1-6). An internal control nucleic acid and a primer set to the internal control were also included as a positive control. The reaction mixture also included reaction buffers, dNTPs, reference dyes, DNA polymerase (TaqGold), and cofactors in concentrations commonly used in the art.

Ten copies of plasmid or MPXV genomic DNA per PCR reaction were able to be detected, as shown in FIGS. 1 and 2, respectively. Both E9L and B6R sets together were found to have combined synergistic effect to increase the overall signal, which was about two-fold higher than those from either of the two sets, thereby increasing the sensitivity of the assay.

Assay sensitivity studies using the described amplification and probe oligonucleotides (SEQ ID NOS: 7-39) and the PCR Cycling Parameters provided below were performed using two commercially available Monkeypox virus cultures purchased from BEI Resources. Monkeypox Virus, WRAIR 7-61 (Catalog No. NR-27) was serially diluted into UTM and amplified using the amplification and probe oligonucleotides described herein. Amplified products were detected at virus dilutions down to 2.8×10\hat{ }0 TCID₅₀/mL. Monkeypox Virus, USA-2003 (Catalog No. NR-2500) was serially diluted into either UTM or a 1:1 mixture of UTM and saliva and amplified using the Monkeypox real-time PCR assay. Amplified products were detected at virus dilutions down to 3.2×10\hat{ }0 pfu/mL in both UTM and UTM/saliva. Therefore, these results demonstrate that assays employing the described amplification and probe oligonucleotides can detect down to 10\hat{ }0 TCID50/mL and 10\hat{ }0 pfu/mL levels of virus which indicates that the assay is sensitive enough to detect the expected minimum infectious levels of monkeypox virus in both UTM and a mixture of UTM and saliva.

PCR Cycling Parameters used in this Example 3 are as follows:

| Stage | Step | Number of cycles | Temperature | | Time (s) |
|---|---|---|---|---|---|
| 1 | 1 | 1 | 92.5 | °C | 600 |
| 2 | 1 | 5 | 92.5 | °C | 30 |
| | 2 | | 63 | °C | 30 |
| 3 | 1 | 45 | 92.5 | °C | 30 |
| | 2 | | 62 | °C | 35 |

### Example 4

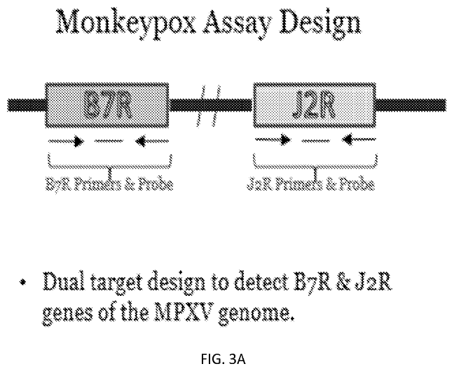

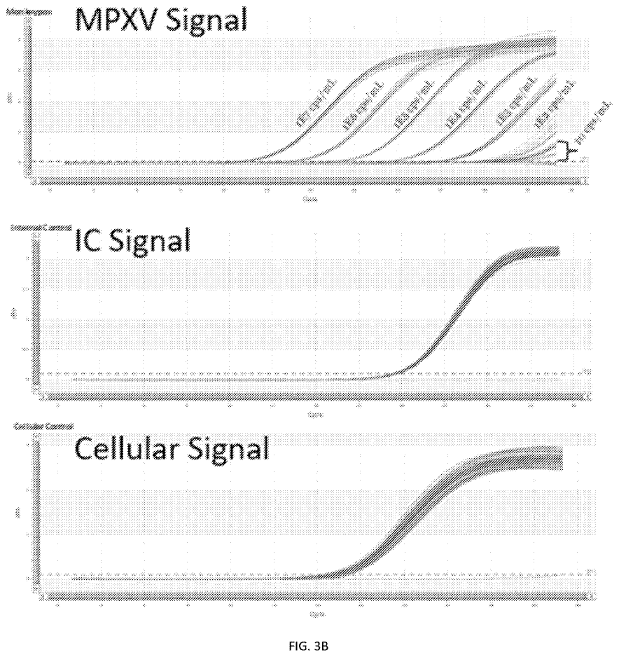

Amplification of monkeypox virus nucleic acid sequences was performed by real-time PCR using the described amplification and probe oligonucleotides. The monkeypox virus sample was provided in the reaction as plasmids carrying B7R and J2R gene (FIG. 3A) The PCR reaction contained the amplification and probe oligonucleotides (SEQ ID NOs: 7, 10, 12, 16, 21 and 26). An internal control nucleic acid, human genomic DNA and sets of amplification and probe oligonucleotides to the internal control and a cellular control were also included as a validity control. The reaction mixture also included reaction buffers, dNTPs, reference dyes, DNA polymerase, and cofactors in concentrations commonly used in the art. The PCR cycling parameters used in this Example 2 are shown below:

| Stage | Step | Number of cycles | Temperature | | Time (s) |
|---|---|---|---|---|---|
| 1 | 1 | 1 | 92.5 | °C | 600 |
| 2 | 1 | 5 | 92.5 | °C | 30 |
| | 2 | | 63 | °C | 30 |
| 3 | 1 | 35 | 92.5 | °C | 30 |
| | 2 | | 62 | °C | 35 |

One hundred copies/mL of MPXV plasmid DNA were able to be detected at 100%, as shown in FIG. 3B.

### Example 5

To evaluate the conservation of oligos designed to detect monkeypox targets in prototype m2000 assays, a sequence analysis pipeline was developed. First, monkeypox complete genome sequences were downloaded from GISAID. All available monkeypox complete genome sequences (678) included in the current release of the standard GISAID MSA (multiple sequence alignment) file were downloaded from GISAID and extracted into a valid fasta file. This download included both Central and Western African lineages (date of download 8/10/22). BLASTn was utilized to produce alignments with each oligo and corrected sequence alignment percent identity was calculated from the local alignment percent identity.

All results are summarized in Table 3 below. The B7R and J2R oligo sets had >90% sequence identity to >99% of all monkeypox sequences analyzed, which included both Central and West African genomes. Therefore, these oligos are predicted to detect most Central and Western African monkeypox infections. In total, two sequences were identified with <90% identity to the SEQ ID NO: 7, one sequence was identified with <90% identity to the SEQ ID NO: 12. For SEQ ID NO: 7, one of the two sequences has incomplete coverage and the other has a deletion at both the 5′ and 3′ end of the oligo. For the SEQ ID NO: 6, the sequence has deletions at both the 5′ and 3′ ends. There are no mismatches for these strains for the J2R oligo set, so the assay would still be able to detect positive results.

TABLE 3
Monkeypox specific oligo inclusivity analysis summary

| | | # with 100% | # with 90-99% | # with <90% | Total |
|---|---|---|---|---|---|
| Target | Oligo | identity | identity | identity | screened |
| monkeypox | SEQ ID NO: 7 | 678 | 0 | 2 | 676 |
| monkeypox | SEQ ID NO: 10 | 678 | 0 | 0 | 678 |
| monkeypox | SEQ ID NO: 12 | 677 | 0 | 1 | 677 |
| monkeypox | SEQ ID NO: 16 | 676 | 0 | 0 | 678 |
| monkeypox | SEQ ID NO: 21 | 678 | 0 | 0 | 678 |
| monkeypox | SEQ ID NO: 26 | 677 | 1 | 0 | 677 |

Oligos directed to B7R are capable of detecting >99% of all Central and Western African monkeypox infections. Oligos directed to J2R are capable of detecting all Central and Western African monkeypox infections. Thus, a dual-target assay utilizing both B7R and J2R oligos are capable of detecting all Central and Western African monkeypox infections.

GISAID Notice: Data, including metadata and sequence information was obtained from the GISAID database as a result of the work of data contributors, i.e., the Authors, the Originating laboratories responsible for obtaining the specimens, and the Submitting laboratories for generating the genetic sequence and metadata and sharing via the GISAID Initiative, on which this research is based. GISAID data are subject to GISAID's Terms and Conditions that can be accessed via gisaid.org. Data cannot be disclosed externally.

### Example 6

To evaluate the potential cross reactivity of monkeypox oligo sets as in Example 3 with off target pathogens, an exclusivity analysis was conducted using BLAST. First, sequences were downloaded from Genbank for each taxon ID and subnodes. Reference sequences and alignments were downloaded from the following: NCBI's NT nucleotide sequence database downloaded on Jun. 29, 2022 (ftp.ncbi.nlm.nih.gov/blast/db/), NCBI's taxonomy database downloaded on Jun. 29, 2022 (ftp.ncbi.nlm.nih.gov/pub/taxonomy/new_taxdump/new_taxdump.tar.gz), NCBI's accession-to-taxid-map database downloaded on Jun. 29, 2022 (ftp.ncbi.nlm.nih.gov/pub/taxonomy/accession2taxid/nucl_gb.accession2taxid.gz), and sequence sets were extracted from the NT database by joining sequence accession to taxids mapped in the nucl_gb.accesion2taxid file. Table 2 (date of accession Jun. 29, 2022) shows the assembled sequences for BLAST alignment processing.

TABLE 4
Count of reference sequences in each Taxid database:

| | | Sequence |
|---|---|---|
| Organism | taxid | Count |
| HIV 1 | 11676 | 1011657 |
| HPV 16 | 333760 | 10514 |
| Mycobacterium tuberculosis | 1773 | 10548 |
| Hepatitis B virus | 10407 | 108951 |
| Escherichia coli | 562 | 111551 |
| Enterococcus faecium | 1352 | 11348 |
| HPV 18 | 333761 | 1190 |
| Human betaherpesvirus 6A | 32603 | 129 |
| Streptococcus salivarius | 1304 | 1325 |
| Streptococcus pneumoniae | 1313 | 13353 |
| Adenovirus type 7 | 10519 | 1353 |
| Neisseria meningitidis | 487 | 13799 |
| Actinomyces israelii | 1659 | 143 |
| Bordetella pertussis | 520 | 1520 |
| Fusobacterium nucleatum | 851 | 1521 |
| Candida albicans | 5476 | 15305 |
| Toxoplasma gondii | 5811 | 16875 |
| Mobiluncus mulieris | 2052 | 17 |
| Acinetobacter calcoaceticus | 471 | 1746 |
| Pseudomonas aeruginosa | 287 | 18356 |
| Staphylococcus saprophyticus | 29385 | 1892 |
| Propionibacterium acnes | 1747 | 2141 |
| Listeria monocytogenes | 1639 | 21726 |
| Staphylococcus aureus | 1280 | 23200 |
| Hepatitis C virus | 11103 | 236422 |
| Treponema pallidum | 160 | 2543 |
| Klebsiella pneumoniae | 573 | 26030 |
| Kingella kingae | 504 | 288 |
| Proteus mirabilis | 584 | 2974 |
| BK virus | 1891762 | 3219 |
| Human betaherpesvirus 6B | 32604 | 386 |
| Neisseria gonorrhoeae | 485 | 4041 |
| Haemophilus ducreyi | 730 | 406 |
| Chlamydia trachomatis serovar D | 272561 | 41 |
| Streptococcus agalactiae | 1311 | 4262 |
| Cryptococcus neoformans | 5207 | 46705 |
| Streptococcus pyogenes | 1314 | 4978 |
| Streptococcus mitis | 28037 | 5099 |
| Moraxella catarrhalis | 480 | 514 |
| Clostridium difficile | 1496 | 5311 |
| Epstein Barr virus | 10376 | 5533 |
| Adenovirus type 1 | 10533 | 563 |
| Chlamydia trachomatis serovar I | 596780 | 6 |
| Staphylococcus epidermidis | 1282 | 6288 |
| Haemophilus influenzae | 727 | 6364 |
| Streptococcus mutans | 1309 | 6690 |
| Enterobacter cloacae | 550 | 6696 |
| Campylobacter jejuni | 197 | 7468 |
| Proteus vulgaris | 585 | 771 |
| Chlamydophila pneumoniae | 83558 | 862 |
| Cytomegalovirus | 10358 | 8967 |
| Clostridium perfringens | 1502 | 9054 |
| Enterococcus faecalis | 1351 | 9440 |
| Acinetobacter lwoffii | 28090 | 983 |
| Bacteroides fragilis | 817 | 993 |

Results were annotated by cross-indexing to the oligo table and the group taxonomy name. Results were sorted by bit scores and the corrected percent identity was calculated by normalizing the length of the alignment to the length of the query oligo. Likewise, the total number of mismatches was corrected to account for end positions that did not align.

The resulting metadata and BLAST results were then processed to generate the ‘midline’ result or the position dots (match) and dashes (mismatch), resulting in >22,000 records. Hits were then parsed to unique oligo target accession values to remove hits that were in the same taxon ID as the intended target. Next, the individual hit with the highest corrected percent identity for each unique oligo/taxon group combination was collected. Results with 79% identity or higher were selected for further manual examination.

For each oligo set the individual oligos with 80% identity or higher were entered into a summary table (Tables 5 and 6 below). Unlisted organisms resulted in no hits for any oligos with >80% identity. Hits where an amplicon was not possible due to only a single oligo having >80% identity were discarded, as well as hits where a forward and reverse oligo had >80% identity but the probe had <80%. Hits where a forward, reverse, and probe all had >80% identity (from Tables 5 and 6) were further examined for a mismatch position in the 3 nucleotides on the 3′ end, for forward and reverse primer being positioned on the same strand, for distance between the forward and reverse oligos, and for probe binding position relative to forward and reverse primers, in an investigation table. Hits in which the forward and reverse oligo were on the same strand, the distance between the forward and reverse oligo were >1000 nucleotides, an insertion or deletion was identified in the alignment, or the probe was not positioned between the forward and reverse primer were predicted not to cause cross-reactivity.

TABLE 5
Exclusivity summary - B7R oligos

| | Parent | SEQ ID | SEQ ID | SEQ ID | |
|---|---|---|---|---|---|
| Organism | tax ID | NO: 7 | NO: 10 | NO: 12 | Set >80% |
| Proteus mirabilis | 584 | NA | 86 | 80 | |
| Candida albicans | 5476 | NA | 86 | 80 | |
| Clostridium difficile | 1496 | 83 | 81 | NA | |
| Escherichia coli | 562 | 83 | 81 | 80 | yes* |
| Klebsiella pneumoniae | 573 | 87 | 86 | NA | |
| Toxoplasma gondii | 5811 | 83 | 86 | 87 | yes* |
| Hepatitis C virus | 11103 | NA | 86 | NA | |
| Moraxella catarrhalis | 480 | NA | 81 | 90 | |
| Enterococcus faecium | 1352 | 83 | 81 | 80 | yes* |
| Streptococcus agalactiae | 1311 | NA | 91 | NA | |
| Enterococcus faecalis | 1351 | 83 | 81 | NA | |
| HIV-1 | 11676 | 83 | 81 | NA | |
| Streptococcus mitis | 28037 | NA | 81 | NA | |
| Enterobacter cloacae | 550 | 87 | 81 | NA | |
| Campylobacter jejuni | 197 | NA | 81 | 80 | |
| Proteus vulgaris | 585 | NA | 81 | 80 | |
| Clostridium perfringens | 1502 | NA | 81 | 80 | |
| Fusobacterium nucleatum | 851 | 83 | 81 | NA | |
| Acinetobacter calcoaceticus | 471 | 83 | 81 | NA | |
| Haemophilus influenzae | 727 | NA | 81 | 83 | |
| Streptococcus salivarius | 1304 | NA | 81 | NA | |
| Cryptococcus neoformans | 5207 | 83 | 81 | 80 | yes* |
| Staphylococcus aureus | 1280 | 83 | 81 | 83 | yes* |
| Staphylococcus epidermidis | 1282 | NA | 81 | 80 | |
| Listeria monocytogenes | 1639 | 91 | 81 | NA | |
| HPV 18 | 333761 | NA | 81 | NA | |
| Streptococcus pneumoniae | 1313 | NA | 81 | NA | |
| Epstein-Barr virus | 10376 | NA | 81 | NA | |
| Staphylococcus saprophyticus | 29385 | NA | 81 | NA | |
| Acinetobacter lwoffii | 28090 | NA | 81 | NA | |
| Pseudomonas aeruginosa | 287 | 83 | 81 | NA | |
| Streptococcus mutans | 1309 | 87 | 81 | NA | |
| Human betaherpesvirus 6A | 32603 | NA | 81 | NA | |
| Mycobacterium tuberculosis | 1773 | NA | 81 | NA | |
| Bacteroides fragilis | 817 | NA | NA | 83 | |

NA for <80%;
*No cross-reaction is predicted per the evaluation criteria

TABLE 6
Exclusivity summary - MPXV-2 A oligos (J2R)

| | Parent | SEQ ID | SEQ ID | SEQ ID | |
|---|---|---|---|---|---|
| Organism | tax ID | NO: 16 | NO: 21 | NO: 26 | Set >80% |
| Klebsiella pneumoniae | 573 | 87 | NA | 86 | |
| Escherichia coli | 562 | 83 | 82 | 86 | yes* |
| Pseudomonas aeruginosa | 287 | 83 | NA | 86 | |
| Staphylococcus aureus | 1280 | 83 | 82 | NA | |
| Acinetobacter calcoaceticus | 471 | 83 | NA | 81 | |
| Enterococcus faecium | 1352 | 83 | NA | NA | |
| HIV-1 | 11676 | 83 | NA | 81 | |
| Clostridium difficile | 1496 | 88 | NA | NA | |
| Toxoplasma gondii | 5811 | 88 | NA | 86 | |
| Propionibacterium acnes | 1747 | 83 | NA | 81 | |
| Hepatitis C virus | 11103 | 83 | NA | 90 | |
| HPV 16 | 333760 | 83 | NA | NA | |
| Neisseria gonorrhoeae | 485 | NA | 82 | 81 | |
| Neisseria meningitidis | 487 | NA | 82 | 86 | |
| Listeria monocytogenes | 1639 | NA | 82 | 81 | |
| Cryptococcus neoformans | 5207 | NA | 82 | 86 | |
| Hepatitis B virus | 10407 | NA | 82 | NA | |
| Enterobacter cloacae | 550 | NA | NA | 86 | |
| Mycobacterium tuberculosis | 1773 | NA | NA | 81 | |
| Streptococcus pyogenes | 1314 | NA | NA | 86 | |
| Bacteroides fragilis | 817 | NA | NA | 90 | |
| Streptococcus agalactiae | 1311 | NA | NA | 81 | |
| Bordetella pertussis | 520 | NA | NA | 81 | |
| Streptococcus mutans | 1309 | NA | NA | 81 | |
| Enterococcus faecalis | 1351 | NA | NA | 81 | |
| Candida albicans | 5476 | NA | NA | 81 | |
| Streptococcus pneumoniae | 1313 | NA | NA | 81 | |

NA for <80%;
*No cross-reaction is predicted per the evaluation criteria

After excluding hits that met the criteria described above, no organisms and oligos included in this analysis were predicted to have cross-reactivity hits.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A set of oligonucleotides for amplifying and detecting monkeypox virus in a sample, comprising:
a first amplification oligonucleotide comprising a nucleic acid sequence with at least 70% similarity to SEQ ID NO: 1, a second amplification oligonucleotide comprising a nucleic acid sequence with at least 70% similarity to SEQ ID NO: 2 and a probe oligonucleotide comprising a nucleic acid sequence with at least 70% similarity to SEQ ID NO: 3; or
a first amplification oligonucleotide comprising a nucleic acid sequence with at least 70% similarity to SEQ ID NO: 4, a second amplification oligonucleotide comprising a nucleic acid sequence with at least 70% similarity to SEQ ID NO: 5, and a probe oligonucleotide comprising a nucleic acid sequence with at least 70% similarity to SEQ ID NO: 6; or
a combination thereof,
wherein each probe oligonucleotide comprises a detectable label.

2. The set of claim 1, wherein the detectable label is a fluorophore.

3. The set of claim 1 or claim 2, wherein each probe oligonucleotide comprises a quencher moiety.

4. The set of claim 1, further comprising primer and probe oligonucleotide sequences for amplifying and detecting an internal control (IC) sequence.

5. A method for detecting monkeypox virus in a sample comprising:
contacting a sample with a set of oligonucleotides of any of claims 1-4 and reagents for amplification;
amplifying one or more target monkeypox virus nucleic acid sequences present in the sample;
hybridizing one or more of the oligonucleotide probes to one or more amplified target monkeypox virus nucleic acid sequences; and
detecting hybridization of the one or more probe oligonucleotide sequences to the one or more amplified monkeypox virus target nucleic acid sequences by measuring a signal from the detectable labels.

6. The method of claim 5, wherein the presence of one or more signals from the detectable label indicates hybridization of the one or more probe oligonucleotides to the one or more amplified monkeypox virus target nucleic acid sequences.

7. The method of claim 5 or claim 6, wherein the reagents for amplification comprise a polymerase, a single-strand binding protein, a helicase, a recombinase, a resolvase, an enzyme cofactor, a buffer, deoxyribonucleotide, or ribonucleotide triphosphates, or combinations thereof.

8. The method of any of claims 5-7, wherein the sample comprises a blood product, an oropharyngeal swab, a rectal swab, a genital swab, saliva, urine, or semen.

9. The method of any of claims 5-7, wherein the sample comprises a skin lesion specimen.

10. A kit for detecting monkeypox virus in a sample comprising:
at least one set of oligonucleotides of any of claims 1-4; or
one or more oligonucleotides comprising a nucleic acid sequence with at least 70% similarity to any of SEQ ID NOs: 1-6.

11. The kit of claim 12, further comprising reagents for amplifying and detecting nucleic acid sequences, and/or instructions for use.

12. The kit of claim 11 or claim 12, further comprising an internal control sequence, primer and probe oligonucleotide sequences for amplifying and detecting an internal control (IC) sequence, or a combination thereof.

13. A system for detecting monkeypox virus in a sample, comprising:
a set of oligonucleotides of any of claims 1-4;
one or more nucleic acid amplification reagents; and
a nucleic acid amplification reaction system configured to amplify one or more target monkeypox virus nucleic acid sequences in the sample.

14. The system of claim 13, wherein the nucleic acid amplification reaction system is further configured to:
facilitate hybridization of one or more of the probe oligonucleotides to amplified target monkeypox virus nucleic acid sequences;
detect hybridization of one or more of the probe oligonucleotides to amplified target monkeypox virus nucleic acid sequences; or
a combination thereof.

15. The system of claim 13 or claim 14, wherein the nucleic acid amplification reaction system is further configured to measure a signal from one or more detectable label.

16. The system of any of claims 13-15, further comprising an internal control sequence, primer and probe oligonucleotide sequences for amplifying and detecting an internal control (IC) sequence, or a combination thereof.

17. The system of any of claims 13-16, wherein the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

18. The system of any of claims 13-17, wherein the sample comprises a blood product, an oropharyngeal swab, a rectal swab, a genital swab, saliva, urine, or semen.

19. The system of any of claims 13-17, wherein the sample comprises a skin lesion specimen.

20. A reaction mixture comprising:
one or more nucleic acid amplification reagents;
a set of oligonucleotides as in any of claims 1-4; and
a sample suspected of containing monkeypox virus.

21. The reaction mixture of claim 20, further comprising an internal control sequence, primer and probe oligonucleotide sequences for amplifying and detecting an internal control (IC) sequence, or a combination thereof.

22. The reaction mixture of claim 20 or claim 21, wherein the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

23. The reaction mixture of any of claims 20-22, wherein the sample comprises a blood product, an oropharyngeal swab, a rectal swab, a genital swab, saliva, urine, or semen.

24. The reaction mixture of any of claims 20-22, wherein the sample comprises a skin lesion specimen.

25. A set of oligonucleotides for amplifying and detecting monkeypox virus in a sample, comprising:
a first amplification oligonucleotide comprising a nucleic acid sequence with at least 70% similarity to any of SEQ ID NOs: 7-9, a second amplification oligonucleotide comprising a nucleic acid sequence with at least 70% similarity to any of SEQ ID NOs: 10-11 and a probe oligonucleotide comprising a nucleic acid sequence with at least 70% similarity to any of SEQ ID NOs: 12-15; or
a first amplification oligonucleotide comprising a nucleic acid sequence with at least 70% similarity to any of SEQ ID NOs: 16-20, a second amplification oligonucleotide comprising a nucleic acid sequence with at least 70% similarity to any of SEQ ID NOs: 21-25, and a probe oligonucleotide comprising a nucleic acid sequence with at least 70% similarity to any of SEQ ID NOs: 26-39; or
a combination thereof,
wherein each probe oligonucleotide comprises a detectable label.

26. The set of claim 25, wherein the detectable label is a fluorophore.

27. The set of claim 25 or claim 26, wherein each probe oligonucleotide comprises a quencher moiety.

28. The set of claim 25, further comprising primer and probe oligonucleotide sequences for amplifying and detecting one or more internal control (IC) sequences.

29. A method for detecting monkeypox virus in a sample comprising:
contacting a sample with a set of oligonucleotides of any of claims 25-28 and reagents for amplification;
amplifying one or more target monkeypox virus nucleic acid sequences present in the sample;
hybridizing one or more of the oligonucleotide probes to one or more amplified target monkeypox virus nucleic acid sequences; and
detecting hybridization of the one or more probe oligonucleotide sequences to the one or more amplified monkeypox virus target nucleic acid sequences by measuring a signal from the detectable labels.

30. The method of claim 29, wherein the presence of one or more signals from the detectable label indicates hybridization of the one or more probe oligonucleotides to the one or more amplified monkeypox virus target nucleic acid sequences.

31. The method of claim 29 or claim 30, wherein the reagents for amplification comprise a polymerase, a single-strand binding protein, a helicase, a recombinase, a resolvase, an enzyme cofactor, a buffer, deoxyribonucleotide, or ribonucleotide triphosphates, or combinations thereof.

32. The method of any of claims 29-32, wherein the sample comprises a blood product, an oropharyngeal swab, a rectal swab, a genital swab, saliva, urine, or semen.

33. The method of any of claims 29-31, wherein the sample comprises a skin lesion specimen.

34. A kit for detecting monkeypox virus in a sample comprising:
at least one set of oligonucleotides of any of claims 25-28; or
one or more oligonucleotides comprising a nucleic acid sequence with at least 70% similarity to any of SEQ ID NOs: 7-39.

35. The kit of claim 34, further comprising reagents for amplifying and detecting nucleic acid sequences, and/or instructions for use.

36. The kit of claim 34 or claim 35, further comprising an internal control sequence, primer and probe oligonucleotide sequences for amplifying and detecting an internal control (IC) sequence, or a combination thereof.

37. A system for detecting monkeypox virus in a sample, comprising:
a set of oligonucleotides of any of claims 25-28;
one or more nucleic acid amplification reagents; and
a nucleic acid amplification reaction system configured to amplify one or more target monkeypox virus nucleic acid sequences in the sample.

38. The system of claim 37, wherein the nucleic acid amplification reaction system is further configured to:
facilitate hybridization of one or more of the probe oligonucleotides to amplified target monkeypox virus nucleic acid sequences;
detect hybridization of one or more of the probe oligonucleotides to amplified target monkeypox virus nucleic acid sequences; or
a combination thereof.

39. The system of claim 37 or claim 38, wherein the nucleic acid amplification reaction system is further configured to measure a signal from one or more detectable label.

40. The system of any of claims 37-39, further comprising an internal control sequence, primer and probe oligonucleotide sequences for amplifying and detecting one or more internal control (IC) sequences, or a combination thereof.

41. The system of any of claims 37-40, wherein the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

42. The system of any of claims 37-41, wherein the sample comprises a blood product, an oropharyngeal swab, a rectal swab, a genital swab, saliva, urine, or semen.

43. The system of any of claims 37-41, wherein the sample comprises a skin lesion specimen.

44. A reaction mixture comprising:
one or more nucleic acid amplification reagents;
a set of oligonucleotides as in any of claims 25-28; and
a sample suspected of containing monkeypox virus.

45. The reaction mixture of claim 44, further comprising an internal control sequence, primer and probe oligonucleotide sequences for amplifying and detecting one or more internal control (IC) sequences, or a combination thereof.

46. The reaction mixture of claim 44 or claim 45, wherein the one or more nucleic acid amplification reagents is selected from the group consisting of: a nicking enzyme, a polymerase, a single-strand binding protein, a recombinase, a helicase, a resolvase, an enzyme cofactor, a buffer, deoxyribonucleotide or ribonucleotide triphosphates, and combinations thereof.

47. The reaction mixture of any of claims 44-46, wherein the sample comprises a blood product, an oropharyngeal swab, a rectal swab, a genital swab, saliva, urine, or semen.

48. The reaction mixture of any of claims 44-46, wherein the sample comprises a skin lesion specimen.
